(19) United States
(12) Patent Application Publication
(10) Pub. No.: US 2012/0190620 A1
(43) Pub. Date: Jul. 26, 2012

(54) METHODS TO IDENTIFY TARGETS AND MOLECULES REGULATING PURINOSOMES AND THEIR USES

(76) Inventors: Stephen Benkovic, State College, PA (US); Ye Fang, Painted Post, NY (US); Songon An, State College, PA (US); Florence Verrier, Corning, NY (US)

(21) Appl. No.: 13/185,018

(22) Filed: Jul. 18, 2011

Related U.S. Application Data
(60) Provisional application No. 61/368,802, filed on Jul. 29, 2010.

(51) Int. Cl. A61K 38/30; A61K 38/18; C07K 14/485; C07C 405/00; A61K 31/5575; A61K 31/662; C07F 9/09; C07D 209/08; A61K 31/4045; C07H 19/20; A61K 31/7076; C07H 19/10; A61K 31/7072; C07K 14/65; C07K 14/475; C12Q 1/02; G01N 21/76; A61K 31/517; A61K 31/506; A61K 31/436; A61K 31/44; A61K 31/404; G01N 33/566; C12Q 1/48; C40B 30/06

(52) U.S. Cl. 514/8.6; 506/10; 514/9.6; 530/300; 562/503; 514/573; 514/120; 558/179; 548/469; 514/415; 536/26.26; 514/47; 536/26.21; 514/51; 514/9.5; 530/397; 435/29; 514/266.4; 514/266.24; 514/252.19; 514/291; 514/346; 514/414; 435/7.21; 435/15

(57) ABSTRACT

The present invention discloses methods to identify targets, pathways and molecules regulating purinosomes and their uses for treating pathophysiological disorders associated with purinosomes. Disclosed are methods related to both label-free cellular assays and fluorescence imaging to confirm the regulatory roles of various targets and molecules in purinosome dynamics. Disclosed are methods to classify molecules and the uses of these molecules for different indications. Specifically, the purinosome-disrupting molecules can be used for improved prevention and treatment of cancer development.

### CROSS REFERENCE TO RELATED APPLICATIONS

This application claims the benefit of priority under 35 U.S.C. §119 of U.S. Provisional Application No. 61/368,802, entitled “Methods to identify targets and molecules regulating purinosomes and their uses”, filed Jul. 29, 2010 the content of which is relied upon and incorporated herein by reference in their entirety.

### BACKGROUND

Purines play many important roles in the life process. Different metabolic pathways exist for: (1) making purines (the synthetic pathways); (2) converting purine compounds (the conversion pathways); (3) reusing purines consumed in the diet (the reuse pathways); and (4) disposing of excess purines (the disposal pathways). As the information molecules in genes, they are used in the process of converting genes to proteins. As energy transducers in cellular signaling processes, such as nerve conduction and muscle contraction, they act as messengers. As a disposal mechanism, they rid cells of excess nitrogen. As antioxidants, they protect the cell from cancer-causing agents.

Purines are not only essential building blocks of DNA and RNA, but, as nucleotide derivatives, they also participate in a multitude of pathways in both prokaryotes and eukaryotes. Biosynthetically, adenosine and guanosine nucleotides are derived from inosine monophosphate (IMP), which is synthesized from phosphoribosyl pyrophosphate (PRPP) in both the de novo and salvage biosynthetic pathways. The salvage pathway catalyzes the one-step conversion of hypoxanthine to IMP by hypoxanthine phosphoribosyl transferase (HPRT), whereas the de novo pathway consists of 10 chemical reactions that transform PRPP to IMP. In higher eukaryotes (such as humans), the de novo pathway uses six enzymes, including three multifunctional enzymes: a trifunctional protein, TrifGART, that has glycinamide ribonucleotide (GAR) synthetase (GARS), GAR transformylase (GAR Tfase), and aminoimidazole ribonucleotide synthetase (AIRS) activities; a bifunctional enzyme, PAICS, that has carboxyaminoimidazole ribonucleotide synthase (CAIRS) and succinylaminoimidazolecarboxamide ribonucleotide synthetase (SAICARS) activities; and a bifunctional enzyme, ATIC, that has aminoimidazolecarboxamide ribonucleotide transformylase (AICAR Tfase) and IMP cyclohydrolase (IMPCH) activities. In contrast, prokaryotes, such as Escherichia coli, use only monofunctional enzymes throughout this pathway, except for the bifunctional ATIC. These enzymes can form a multienzyme complex, depending on cellular status. The association and dissociation of the multienzyme complex can be regulated by cellular purine levels that were imposed by the addition of external reagents that regulate purine metabolic flux. These functional complexes may produce efficient substrate channels that link the 10 catalytic active sites. Additionally, clustering of the 10 active sites may provide an efficient means of globally regulating purine flux under varying environmental conditions. These multienzyme complexes observed in the de novo purine biosynthetic pathway can constitute a “purinosome.” The formation of the purinosome appears to be dynamically regulated by stimulation of de novo purine biosynthesis in response to changes in purine levels. The purinosome may be a general phenomenon in all cell types during specific stages of the cell cycle, along with posttranslation modifications. Because of the relevance of de novo purine biosynthesis to human diseases, the purinosome may represent a new pharmacological opportunity for therapeutic intervention.

Several inhibitors of protein kinases have been approved or entered clinical trials for treatment of cancer. Generally, protein kinases catalyze the phosphorylation of tyrosine, serine, or threonine residues on proteins using ATP as a substrate. Many of the kinase inhibitors that are approved or under development are competitive inhibitors with respect to ATP. Thus, the effectiveness of the protein kinase inhibitor is sensitive to the concentration of cellular ATP. The efficacy and activity of protein kinase inhibitors could be maximized if cellular ATP levels could easily be manipulated. This invention solves this and other problems.

### SUMMARY

Disclosed are methods of identifying cellular targets involved in regulating purinosome dynamics comprising a) providing a cell, b) contacting the cell having a known cellular target with a molecule having a known cellular target forming a molecule incubated cell, c) contacting the molecule incubated cell sequentially with a purinosome promoting agent and a purinosome disrupting agent, d) monitoring the response of the molecule incubated cell after contact with the purinosome promoting agent and after contact with the purinosome disrupting agent, and e) determining the ability of the molecule to modulate the dynamics of the purinosome formation and dissociation.

Disclosed are methods of identifying a purinosome dynamics modulating pathway comprising a) providing a cell, b) contacting the cell having a known cellular target with a cellular pathway modulator specific to the cellular pathway of said cellular target to obtain a cellular pathway modulator incubated cell, c) contacting the cellular pathway modulator incubated cell with a ligand specific to said cellular target to obtain the cellular pathway modulator and ligand incubated cell, d) contacting said cellular pathway modulator and ligand incubated cell with a purinosome modulating agent, e) assaying the response of the cell; and f) determining the ability of the cellular pathway modulator to regulate purinosome dynamics, wherein the ability of the cellular pathway modulator to regulate purinosome dynamics indicates the cellular pathway is a purinosome dynamics modulating pathway.

Disclosed are methods of identifying purinosome dynamics modulators comprising a) providing a cell, b) contacting the cell with a molecule forming a molecule incubated cell, c) contacting the molecule incubated cell sequentially with a purinosome promoting agent and a purinosome disrupting agent, d) monitoring the response of the molecule incubated cell after contact with the purinosome promoting agent and after contact with the purinosome disrupting agent, and e) determining the ability of the molecule to modulate the dynamics of the purinosome formation and dissociation.

Disclosed are methods of treating a subject comprising administering to the subject a therapeutically effective amount of a purinosome dynamics modulator, wherein the subject has a disease which is pathophysiologically related to purinosomes.

Disclosed are pharmaceutical compositions for treating a subject comprising a therapeutically effective amount of a purinosome dynamics modulating molecule.

In some forms of the disclosed methods, the purinosome promoting agent contacts the cell before the purinosome disrupting agent. In some forms, the purinosome disrupting agent contacts the cell before the purinosome promoting agent.

In some forms of the disclosed methods, the ability of the molecule to modulate the dynamics of the purinosome formation and dissociation indicates the molecule's known cellular target has a role in regulating the purinosome dynamics.

In some forms, the disclosed methods further comprise classifying the cellular targets and the purinosome dynamics modulators based on their regulation of purinosome dynamics.

In some forms of the disclosed methods, the classifying of cellular targets and purinosome dynamics modulators can be based on correlation analysis. The correlation analysis can be done between a purinosome disrupting agent response and a purinosome promoting agent response.

In some forms of the disclosed methods, the classifying of cellular targets and purinosome dynamics modulators can be based on similarity analysis. The similarity analysis can be done by Hierarchy Euclidean clustering of the purinosome disrupting agent's response and the purinosome promoting agent's response in the cell.

In some forms of the disclosed methods, the molecule stimulated cellular target or the purinosome dynamics modulator can potentiate the purinosome promoting agent's response and have little or no effect on the purinosome disrupting agent's response.

In some forms of the disclosed methods, the molecule stimulated cellular target or the purinosome dynamics modulator selectively suppresses the purinosome promoting agent's response.

In some forms of the disclosed methods, the molecule stimulated cellular target or the purinosome dynamics modulator suppresses the purinosome promoting agent's response and potentiates the purinosome disrupting agent's response.

In some forms of the disclosed methods, the molecule stimulated cellular target or purinosome dynamics modulator has no effect.

In some forms of the disclosed methods, the molecule stimulated cellular target or purinosome dynamics modulator selectively suppresses the purinosome disrupting agent's response.

In some forms of the disclosed methods, the molecule stimulated cellular target or purinosome dynamics modulator selectively potentiates the purinosome promoting agent's response.

In some forms of the disclosed methods, the molecule stimulated cellular target or purinosome dynamics modulator selectively potentiates the purinosome disrupting agent's response.

In some forms of the disclosed methods, the molecule stimulated cellular target or the purinosome dynamics modulator potentiates the purinosome promoting agent's response and suppresses the purinosome disrupting agent's response.

In some forms of the disclosed methods, the purinosome promoting agent can be a purinosome-promoting CK2 inhibitor. The purinosome-promoting CK2 inhibitor can be DMAT.

In some forms of the disclosed methods, the purinosome modulating agent can be a purinosome disrupting agent. The purinosome disrupting agent can be a purinosome-disrupting CK2 inhibitor. The purinosome-disrupting CK2 inhibitor can be TBB.

In some forms of the disclosed methods, the monitoring of the response can be performed with a label-free biosensor cellular assay. In some forms, the monitoring of the response can be performed with fluorescent imaging.

In some forms of the disclosed methods, the molecule can be an agonist.

In some forms of the disclosed methods, the cellular target can be a G protein-coupled receptor (GPCR), a receptor tyrosine kinase, a Toll-like receptor, a cytokine receptor, or ion channel. The GPCR can be a prostaglandin receptor, serotonin receptor, adrenergic receptor (AR), lysophosphatidic acid (LPA) receptor, P2Y2 receptor, or a sphingosine 1-phosphate (S1P) receptor. In some forms, the cell can be a HeLa cell line. In some forms, the prostaglandin receptor can be EP4, the serotonin receptor can be 5HT1D, the adrenergic receptor can be alpha2A-AR, the LPA receptor can be LPA 1, 2 or 5 and the S1P receptor can be S1P 2, 3, or 5.

In some forms of the disclosed methods, determining the ability of the cellular pathway modulator to regulate purinosome dynamics comprises comparing the effects of the cellular pathway modulator, ligand, and purinosome modulating agent incubated cell to a control.

In some forms of the disclosed methods, the ligand can be an agonist.

In some forms of the disclosed methods, the control can be a ligand and purinosome modulating agent incubated cell without a cellular pathway modulator.

In some forms of the disclosed methods, the cellular pathway modulator can be interference RNA, or a kinase inhibitor. The cellular pathway modulator can be a toxin. In some forms, the toxin can be pertussis toxin or cholera toxin.

In some forms of the disclosed methods, the ability of the molecule to modulate the dynamics of the purinosome formation and dissociation indicates the molecule can be a purinosome dynamics modulator.

In some forms, the disclosed methods further comprise one or more therapeutic agents. In some forms, the therapeutic agents can be an anti-cancer agent. In some forms, the therapeutic agent can be a protein kinase inhibitor. The protein kinase inhibitor can be selected from erlotinib, lapatinib, dasatinib, temsirolimus, rapamycin, sorafenib, or sunitinib.

In some forms of the disclosed methods, the purinosome dynamics modulator and the therapeutic agent produce a synergistic effect.

In some forms of the disclosed methods, the purinosome dynamics modulator can be a purinosome-disrupting modulator.

In some forms of the disclosed methods, the disease can be selected from the group consisting of cancer, inflammatory diseases, and metabolic disorders. In some forms, the cancer can be selected from the group consisting of leukemias, prostate cancer, hormone dependent cancers, breast cancer, colon cancer, ovarian, cancer, lung cancer, epidermal cancer, liver cancer, esophageal cancer, stomach cancer, brain cancer, and cancer of the kidney.

In some forms of the disclosed methods, the purinosome dynamics modulator targets a GPCR pathway. In some forms, the GPCR pathway can be an adrenergic receptor pathway. The adrenergic receptor pathway can be the alpha2A adrenergic receptor pathway.

In some forms of the disclosed methods, the purinosome dynamics modulator can be an alpha2A adrenergic receptor antagonist. The alpha2A adrenergic receptor antagonist can be Yohimbine.

In some forms of the disclosed methods, the purinosome dynamics modulator can be a receptor ligand that can reverse the effects of an actively signaling receptor.

In some forms of the disclosed methods, the purinosome dynamics modulator and the therapeutic agent can be administered concurrently or sequentially. In some forms, the purinosome dynamics modulator and the therapeutic agent can be administered by the same or different routes.

In some forms of the disclosed methods, the purinosome dynamics modulator or therapeutic agent alone have no effect on the disease.

In some forms of the disclosed methods, the subject can be a mammal.

In some forms, the methods further comprise identifying the subject as needing treatment for the disease. In some forms, the methods further comprise monitoring the subject for therapeutic efficacy. In some forms, monitoring the subject for efficacy of the treatment comprises analyzing a sample obtained from the subject.

In some forms, the disclosed compositions further comprise one or more therapeutic agents. The therapeutic agent can be an anti-cancer agent.

In some forms, the purinosome dynamics modulating molecule and the therapeutic agent produce a synergistic effect in treating diseases which are pathophysiologically related to purinosomes.

In some forms, the subject can be a mammal.

### BRIEF DESCRIPTION OF FIGURES

FIG. 1 represents schematic flowcharts (A and B) of disclosed methods to identify cellular targets and pathways and molecules that regulate the purinosome dynamics. Both flowcharts also represent the disclosed methods which are useful to classify the molecules in terms of their ability to modulate the purinosome dynamics.

FIG. 2 shows the dynamics of CK2 inhibitor-induced DMR signals of HeLa cells. (A) The purinosome disrupting CK2 inhibitor TBB induced DMR signal was reversed by the subsequent stimulation with DMAT, a purinosome promoting CK2 inhibitor. (B) The CK2 inhibitor DMAT induced DMR signal was also reversed by the subsequent stimulation with TBB. All cells were cultured on Epic® tissue culture compatible 384 well microplates for 1 day using regular serum medium with an initial seeding density of 25K cells per well. Under all conditions, the TBB concentration was 50 μM, while the DMAT concentration was 25 μM. At least 4 replicates for each were used to generate the average responses.

FIG. 3 shows a screening result showing certain cellular targets whose signaling regulates purinosome dynamics. The amplitude of the DMAT DMR signal (20 min post stimulation) is correlated with the amplitude of the TBB DMR signal (50 min post stimulation) in the presence of different molecules. A library of agonists for various different GPCRs were used to individually pretreat a confluent layer of Hela cells for about one hour, before sequential stimulation with DMAT and TBB, respectively. The responses were monitored using Epic® system.

FIG. 4 shows a screening result showing certain cellular targets whose signaling regulates purinosome dynamics. The amplitude of the TBB DMR signal (50 min post stimulation) is correlated with the amplitude of the DMAT DMR signal (20 min post stimulation) in the presence of different molecules. A library of agonists for various different GPCRs were used to individually pretreat a confluent layer of Hela cells for about one hour, before sequential stimulation with TBB and DMAT, respectively. The responses were monitored using Epic® system.

FIG. 5 shows a screening result showing certain molecules that regulate purinosome dynamics. The amplitude of the DMAT DMR signal (20 min post stimulation) is correlated with the amplitude of the TBB DMR signal (50 min post stimulation) in the presence of different molecules. A library of molecules targeting endogenous alpha2A and beta2 adrenergic receptors were used to individually pretreat a confluent layer of Hela cells for about one hour, before sequential stimulation with DMAT and TBB, respectively. The responses were monitored using Epic® system.

FIG. 6 represents purinosome dynamics. (A) Confocal fluorescence image of an A549 cell cultured under the purine-rich medium. (B) Confocal fluorescence image of the same A549 cell in (A) after stimulated with DMAT. (C) Confocal fluorescence image of an A549 cell cultured under the purine-depleted medium. (D) Confocal fluorescence image of the same A549 cell in (C) after stimulation with TBB. The fluorescence is due to the expression of GFP-tagged hFGAMS, an enzyme involved in purine synthesis pathway and purinosome complex. The appearance of fluorescent clusters indicates the formation of purinosome complexes; vice versa, the diffusive fluorescent pattern indicates no or little purinosome complexes formed.

FIG. 7 represents the linkage of signaling of endogenous alpha2A adrenergic receptor, but not endogenous beta2 adrenergic receptor, with the purinosome formation (A and C). Oxymetazoline (B) is an alpha2A AR-specific agonist, while salmeterol (D) is a beta2AR-specific agonist. Hela cells were cultured using purine rich medium (10% fetal calf serum). The fluorescence is due to the expression of GFP-tagged hFGAMS, an enzyme involved in purine synthesis pathway and purinosome complex. The appearance of fluorescent clusters indicates the formation of purinosome complexes; vice versa, the diffusive fluorescent pattern indicates no or little purinosome complexes formed.

FIG. 8 represents the ability of alpha2A adrenergic receptor antagonist Yohimbine to reverse the alpha2A receptor agonist oxymetazoline induced purinosome complexes in HeLa cell. (A) Untreated; (B) stimulated with oxymetazoline; and (C) subsequently stimulated with yohimbine. Hela cells were cultured using purine rich medium (10% fetal calf serum). The fluorescence is due to the expression of GFP-tagged hFGAMS, an enzyme involved in purine synthesis pathway and purinosome complex. The appearance of fluorescent clusters indicates the formation of purinosome complexes; vice versa, the diffusive fluorescent pattern indicates no or little purinosome complexes formed.

FIG. 9 shows the pathways involved in regulating purinosome dynamics. (A) The dose-dependent DMR signals of Hela cells induced by alpha2A agonist clonidine. (B) The TBB DMR signal of Hela cells after pretreated with clonidine at different doses. (C) The dose-dependent DMR signals of pertussis toxin (PTX)-pretreated Hela cells induced by alpha2A agonist clonidine. (D) The TBB DMR signal of the PTX-pretreated Hela cells after stimulated with clonidine at different doses. The TBB concentration was 20 μM in both (C) and (D). The clonidine pretreatment occurred about one-hour before the TBB stimulation.

### DETAILED DESCRIPTION

### A. Background

1. Purinosomes

Proteins are likely to organize into complexes that assemble and disassemble depending on cellular needs. In a quiescent state of yeast cells, a large number of proteins involved in intermediary metabolism and stress response were observed to form punctate cytoplasmic foci. The purine biosynthetic enzyme Ade4-GFP formed foci in the absence of adenine, and cycling between punctate and diffuse phenotypes could be controlled by adenine subtraction and addition. Similarly, glutamine synthetase (Gln1-GFP) foci cycled reversibly in the absence and presence of glucose. The structures were neither targeted for vacuolar or autophagosome degradation nor colocalized with P bodies or major organelles. Thus, upon nutrient depletion, the cell induces widespread protein assemblies displaying nutrient-specific formation and dissolution.

A recent study using fluorescence microscopy to HeLa cells (An, S. et al. “Reversible compartmentalization of de novo purine biosynthetic complexes in living cells”. Science 2008, 320: 103-106) indicates that all six enzymes related to purine synthesis pathway colocalize to form clusters in the cellular cytoplasm. The association and dissociation of these enzyme clusters can be regulated dynamically, by either changing the purine levels of or adding exogenous agents to the culture media. This finding provides strong evidence for the formation of a multi-enzyme complex, the “purinosome,” to carry out de novo purine biosynthesis in cells.

CK2 and Akt (also known as protein kinase B) have been implicated to interact with de novo purine biosynthetic enzymes based on two different in vitro proteomic scale experiments; i) hPPAT, hTrifGART and hFGAMS are substrates for hCK2, and ii) hFGAMS is a substrate for Akt. Several key metabolic enzymes were described to be implicated as substrates of CK2 or Akt; for example, glycogen synthase, acetyl-CoA carboxylase and ornithine decarboxylase for CK2, and ATP-citrate lyase for Akt.

### B. Methods

1. Identifying/Screening

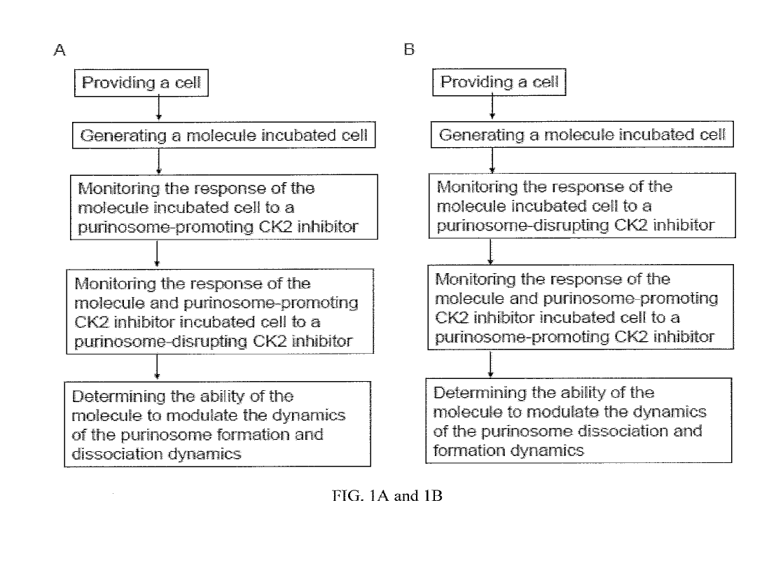

Disclosed are methods to identify cellular targets, pathways and molecules that regulate the purinosome dynamics. Disclosed methods, as shown in FIG. 1, comprise (a) Providing a cell, (b) Generating a molecule incubated cell, (c) Monitoring the response of the molecule incubated cell to a purinosome-promoting CK2 inhibitor, (d) Monitoring the response of the molecule and purinosome-promoting CK2 inhibitor incubated cell to a purinosome-disrupting CK2 inhibitor, (e) Determining the ability of the molecule to modulate the dynamics of the purinosome formation and dissociation dynamics, wherein the steps (c) and (d) involve the use of fluorescence imaging of a fluorescent enzyme that is part of the purinosome complex, and/or the use of a label-free biosensor-based whole cell sensing.

Disclosed is also an alternative method comprising (a) providing a cell, (b) generating a molecule incubated cell, (c) monitoring the response of the molecule incubated cell to a purinosome-disrupting CK2 inhibitor, (d) monitoring the response of the molecule and purinosome-promoting CK2 inhibitor incubated cell to a purinosome-promoting CK2 inhibitor, (e) determining the ability of the molecule to modulate the dynamics of the purinosome dissociation and formation dynamics, wherein the steps (c) and (d) involve the use of fluorescence imaging of a fluorescent enzyme that is part of the purinosome complex, and/or the use of a label-free biosensor-based whole cell sensing.

a) Cellular Targets

Disclosed are methods of identifying cellular targets involved in regulating purinosome dynamics comprising a) providing a cell, b) contacting the cell having a known cellular target with a molecule having a known cellular target forming a molecule incubated cell, c) contacting the molecule incubated cell sequentially with a purinosome promoting agent and a purinosome disrupting agent, d) monitoring the response of the molecule incubated cell after contact with the purinosome promoting agent and after contact with the purinosome disrupting agent, and e) determining the ability of the molecule to modulate the dynamics of the purinosome formation and dissociation.

In some forms of the disclosed methods, the purinosome promoting agent contacts the cell before the purinosome disrupting agent. In some forms, the purinosome disrupting agent contacts the cell before the purinosome promoting agent. In either case, the time between the cell contact of the purinosome promoting agent and the purinosome disrupting agent can be, but is not limited to, 1, 5, 10, 15, 30, or 60 minutes. The time between the cell contact of the purinosome promoting agent and the purinosome disrupting agent can be, but is not limited to, 1, 2, 3, 4, 5, 10, 12, 18, or 24 hours.

In some forms of the disclosed methods, the ability of the molecule to modulate the dynamics of the purinosome formation and dissociation indicates the molecule's known cellular target has a role in regulating the purinosome dynamics.

In some forms of the disclosed methods, the purinosome promoting agent can be a purinosome-promoting CK2 inhibitor. The purinosome-promoting CK2 inhibitor can be DMAT.

In some forms of the disclosed methods, the purinosome disrupting agent can be a purinosome-disrupting CK2 inhibitor. The purinosome-disrupting CK2 inhibitor can be TBB.

In some forms of the disclosed methods, the monitoring of the response can be performed with a label-free biosensor cellular assay. In some forms, the monitoring of the response can be performed with fluorescent imaging.

In some forms of the disclosed methods, the molecule can be an agonist.

In some forms of the disclosed methods, the cellular target can be a G protein-coupled receptor (GPCR), a receptor tyrosine kinase, a Toll-like receptor, a cytokine receptor, or ion channel. The GPCR can be a prostaglandin receptor, serotonin receptor, adrenergic receptor (AR), lysophosphatidic acid (LPA) receptor, P2Y2 receptor, or a sphingosine 1-phosphate (S1P) receptor. In some forms, the cell can be a HeLa cell line. In some forms, the prostaglandin receptor can be EP4, the serotonin receptor can be 5HT1D, the adrenergic receptor can be alpha2A-AR, the LPA receptor can be LPA 1, 2 or 5 and the S1P receptor can be S1P 2, 3, or 5.

Purinosome formation can be a downstream event of many GPCR signaling, and the regulation of purinosome complexes represents an immediate response of GPCR signaling, compared to the late regulation of gene expression activated by GPCR signaling. Thus, the regulation of purinosome dynamics represents a missing link between certain GPCRs and their roles in mitogenic responses. Certain GPCR antagonists can be effective in inhibiting purinosome formation under pathophysiological conditions, indicating that these antagonists can be used as a class of effective chemotherapy agents.

Beside GPCRs, many other classes of receptors can also play important roles in regulating purinosome dynamics. Examples are, but are not limited to, cell surface membrane associated receptor tyrosine kinases, Toll-like receptors, ion channels and intracellular enzymes and kinases.

b) Cellular Pathways

Disclosed are methods of identifying a purinosome dynamics modulating pathway comprising a) providing a cell, b) contacting the cell having a known cellular target with a cellular pathway modulator specific to the cellular pathway of said cellular target to obtain a cellular pathway modulator incubated cell, c) contacting the cellular pathway modulator incubated cell with a ligand specific to said cellular target to obtain the cellular pathway modulator and ligand incubated cell, d) contacting said cellular pathway modulator and ligand incubated cell with a purinosome modulating agent, e) assaying the response of the cell, and f) determining the ability of the cellular pathway modulator to regulate purinosome dynamics, wherein the ability of the cellular pathway modulator to regulate purinosome dynamics indicates the cellular pathway is a purinosome dynamics modulating pathway.

In some forms of the disclosed methods, the ligand can be an agonist. The ligand can be the known specific ligand for the cellular target or can be a molecule that mimics the ligand and can directly or indirectly activate the cellular target's pathway.

In some forms of the disclosed methods, the purinosome modulating agent can be a purinosome disrupting agent. In some forms, the purinosome disrupting agent can be a purinosome disrupting CK2 inhibitor. The purinosome disrupting CK2 inhibitor can be TBB.

In some forms of the disclosed methods, determining the ability of the cellular pathway modulator to regulate purinosome dynamics comprises comparing the effects of the cellular pathway modulator, ligand, and purinosome modulating agent incubated cell to a control. In some forms, the control can be a ligand and purinosome modulating agent incubated cell without a cellular pathway modulator.

In some forms of the disclosed methods, the cellular pathway modulator can be interference RNA, or a kinase inhibitor. In some forms, the cellular pathway modulator can be a toxin. For example, the toxin can be pertussis toxin or cholera toxin. The cellular pathway modulator can affect different stages of the pathway. For instance, some cellular pathway modulators can affect the early stages of the pathway while other modulators may affect a downstream signaling event of the pathway. If a signaling via a cellular target starts as one pathway and then splits into two or more different pathways, it can be useful to use cellular pathway modulators specific to each divergent pathway.

c) Purinosome Dynamics Modulators

Disclosed are methods of identifying purinosome dynamics modulators comprising a) providing a cell, b) contacting the cell with a molecule forming a molecule incubated cell, c) contacting the molecule incubated cell sequentially with a purinosome promoting agent and a purinosome disrupting agent, d) monitoring the response of the molecule incubated cell after contact with the purinosome promoting agent and after contact with the purinosome disrupting agent, and e) determining the ability of the molecule to modulate the dynamics of the purinosome formation and dissociation.

In some forms of the disclosed methods, the purinosome promoting agent contacts the cell before the purinosome disrupting agent. In some forms, the purinosome disrupting agent contacts the cell before the purinosome promoting agent. In either case, the time between the administration of the first agent and the second agent can be, but is not limited to, 1, 5, 10, 15, 30, or 60 minutes. The time between the administration of the first agent and the second agent can be, but is not limited to, 1, 2, 3, 4, 5, 10, 12, 18, or 24 hours.

In some forms of the disclosed methods, the ability of the molecule to modulate the dynamics of the purinosome formation and dissociation indicates the molecule can be a purinosome dynamics modulator.

In some forms of the disclosed methods, the purinosome promoting agent can be a purinosome-promoting CK2 inhibitor. The purinosome-promoting CK2 inhibitor can be DMAT.

In some forms of the disclosed methods, the purinosome disrupting agent can be a purinosome-disrupting CK2 inhibitor. The purinosome-disrupting CK2 inhibitor can be TBB.

In some forms of the disclosed methods, the monitoring of the response can be performed with a label-free biosensor cellular assay. In some forms, the monitoring of the response can be performed with fluorescent imaging.

2. Classifying

a) Cellular Targets

In some forms, the methods further comprise classifying the cellular targets based on their regulation of purinosome dynamics.

In some forms of the methods, the classifying of cellular targets can be based on correlation analysis. The correlation analysis can be done between a purinosome disrupting agent response and a purinosome promoting agent response.

In some forms of the methods, the classifying of cellular targets can be based on similarity analysis. The similarity analysis can be done by Hierarchy Euclidean clustering of the purinosome disrupting agent's response and the purinosome promoting agent's response in the cell.

In some forms of the methods, the molecule stimulated cellular target potentiates the purinosome promoting agent's response and has little or no effect on the purinosome disrupting agent's response.

In some forms of the disclosed methods, the molecule stimulated cellular target selectively suppresses the purinosome promoting agent's response.

In some forms of the disclosed methods, the molecule stimulated cellular target suppresses the purinosome promoting agent's response and potentiates the purinosome disrupting agent's response.

In some forms of the disclosed methods, the molecule stimulated cellular target has no effect.

In some forms of the disclosed methods, the molecule stimulated cellular target selectively suppresses the purinosome disrupting agent's response.

In some forms of the disclosed methods, the molecule stimulated cellular target selectively potentiates the purinosome promoting agent's response.

In some forms of the disclosed methods, the molecule stimulated cellular target selectively potentiates the purinosome disrupting agent's response.

b) Purinosome Dynamics Modulator

In some forms, the disclosed methods further comprise classifying the purinosome dynamics modulator based on its regulation of purinosome dynamics. In some forms, the classifying of the purinosome dynamics modulator can be based on correlation analysis. The correlation analysis can be done between a purinosome disrupting agent response and a purinosome promoting agent response. In some forms, the classifying of the purinosome dynamics modulator can be based on similarity analysis. The similarity analysis can be done by Hierarchy Euclidean clustering of the purinosome disrupting agent's response and the purinosome promoting agent's response in the cell.

In some forms of the disclosed methods, the purinosome dynamics modulator potentiates the purinosome promoting agent's response and has little or no effect on the purinosome disrupting agent's response.

In some forms of the disclosed methods, the purinosome dynamics modulator selectively suppresses the purinosome promoting agent's response.

In some forms of the disclosed methods, the purinosome dynamics modulator suppresses the purinosome promoting agent's response and potentiates the purinosome disrupting agent's response.

In some forms of the disclosed methods, the purinosome dynamics modulator has no effect.

In some forms of the disclosed methods, the purinosome dynamics modulator selectively suppresses the purinosome disrupting agent's response.

In some forms of the disclosed methods, the purinosome dynamics modulator selectively potentiates the purinosome promoting agent's response.

In some forms of the disclosed methods, the purinosome dynamics modulator selectively potentiates the purinosome disrupting agent's response.

In some forms of the disclosed methods, the purinosome dynamics modulator potentiates the purinosome promoting agent's response and suppresses the purinosome disrupting agent's response.

Disclosed are also methods to classify a cellular target, a pathway, or a molecule for their ability to modulate purinosome dynamics, comprising (a) determining the ability of the molecule to modulate the purinosome dynamics, and (b) classifying the molecule into different categories, based on correlation analysis wherein the correlation analysis is done between the purinosome-disrupting CK2 inhibitor DMR and the purinosome-promoting CK2 inhibitor DMR, wherein the two CK2 inhibitors are used to sequentially stimulate the molecule incubated cell. This method further comprises the examination of fluorescent pattern of a fluorescent enzyme that is part of the purinosome complex, wherein the fluorescent enzyme is introduced inside the cell, wherein the introduction is taken place via gene transfection and expression, or protein delivery.

Disclosed are methods to classify a cellular target, a pathway, or a molecule for their ability to modulate purinosome dynamics, comprising (a) determining the ability of the molecule to modulate the purinosome dynamics, and (b) classifying the molecule into different categories, based on similarity analysis wherein the similarity analysis is done by Hierarchy Euclidean clustering of the purinosome-disrupting CK2 inhibitor DMR and the purinosome-promoting CK2 inhibitor DMR in the molecule incubated cell, wherein the two CK2 inhibitors are used to individually or sequentially stimulate the molecule incubated cell. The two different orders can be used for the sequential simulation.

3. Treating

Disclosed are methods of treating a subject comprising administering to the subject a therapeutically effective amount of a purinosome dynamics modulator, wherein the subject has a disease which is pathophysiologically related to purinosomes.

In some forms, the disclosed methods further comprise one or more therapeutic agents. The therapeutic agent can be an anti-cancer agent, anti-inflammatory agent or anti-angiogenic agent. In some forms, the therapeutic agent can be a protein kinase inhibitor. The protein kinase inhibitor can be selected from erlotinib, lapatinib, dasatinib, temsirolimus, rapamycin, sorafenib, or sunitinib. The protein kinase inhibitor can be a CK2 inhibitor.

In some forms of the disclosed methods, the purinosome dynamics modulator and the therapeutic agent can produce a synergistic effect. In some forms, the effect is a combination of the modulator's and agent's individual effects.

In some forms of the disclosed methods, the purinosome dynamics modulator can be a purinosome-disrupting modulator.

In some forms of the disclosed methods, the disease can be selected from the group consisting of cancer, inflammatory diseases, and metabolic disorders. The cancer can be selected from the group consisting of leukemias, prostate cancer, hormone dependent cancers, breast cancer, colon cancer, ovarian, cancer, lung cancer, epidermal cancer, liver cancer, esophageal cancer, stomach cancer, brain cancer, and cancer of the kidney.

In some forms of the disclosed methods, the purinosome dynamics modulator can target a GPCR pathway. The GPCR pathway can be an adrenergic receptor pathway. In some forms, the adrenergic receptor pathway can be the alpha2A adrenergic receptor pathway.

In some forms of the disclosed methods, the purinosome dynamics modulator can be an alpha2A adrenergic receptor antagonist. The alpha2A adrenergic receptor antagonist can be Yohimbine.

In some forms of the disclosed methods, the purinosome dynamics modulator can be a receptor ligand that can reverse the effects of an actively signaling receptor.

In some forms of the disclosed methods, the purinosome dynamics modulator and the therapeutic agent can be administered concurrently or sequentially. The time between the sequential administration of the purinosome dynamics modulator and the therapeutic agent can be 1, 5, 10, 15, 30, or 60 minutes. It can also be 2, 3, 4, 5, 6, 7, 8, 9, 10, 12, 18, 24, 48 or 72 hours. In some forms, the sequential administration of the therapeutic agent can occur any time after the administration of purinosome dynamics modulator as long as the modulator or the effects are still present in the subject.

In some forms of the disclosed methods, the purinosome dynamics modulator and the therapeutic agent can be administered by the same or different routes.

In some forms of the disclosed methods, the purinosome dynamics modulator or therapeutic agent alone have no effect on the disease. However, the combination of the two can have a wide range of effects on the disease such as, eliminating one or more symptoms, increasing the overall health of the subject, or completely eliminating all disease-associated complications.

In some forms of the disclosed methods, the subject can be a mammal. In some forms, the methods further comprise identifying the subject as needing treatment for the disease. A subject needing treatment for a disease can be a subject who has been previously diagnosed with the disease or is simply exhibiting symptoms associated with the disease. A subject in need of treatment for a disease can be a subject at risk for the disease.

In some forms, the disclosed methods further comprise monitoring the subject for therapeutic efficacy. Monitoring the subject for efficacy of the treatment can comprise analyzing a sample obtained from the subject. The sample can be, but is not limited to, a bodily fluid, such as but not limited to, urine, blood, plasma and serum, or a tissue, muscle, hair, organ, or cell.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a component effective to yield the desired therapeutic response, for example, an amount effective to delay the growth of a cancer or to cause a cancer to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

The disclosed methods provide methods of treating cancer using purinosome disrupting inhibitors. Purinosome formation is important for de novo purine synthesis, and purine synthesis pathway inhibitors are viable approach for anti-cancer therapy. It is worthy noting that many purine synthesis pathway inhibitors do not involve in regulation of purinosome complexes (data not shown).

This invention also provides novel compositions and methods to treat cancer using a purinosome disrupting inhibitor, combined with a known oncogenic drug. A known oncogenic drug includes drugs that target mitosis, DNA synthesis and repair systems (e.g., microtubule binding drugs such as vinblastine and vincristine), or drugs that target membrane-bound receptor kinases (HGF/c-Met, EGFR, IGF-1R, PDGFR) or intracellular signaling kinases (Src, PI3k/Akt/mTOR, and MAPK pathways), or drugs that target epigenetic abnormalities (DNA methyltransferase and histone deacetylase), or drugs that target protein dynamics (heat shock protein 90, ubiquitin-proteasome system) or tumor vasculature and microenvironment (angiogenesis, HIF, endothelium, integrins).

This invention also provides novel compositions and methods to treat cancer using a purinosome disrupting inhibitor, combined with a protein kinase inhibitor.

In one embodiment, cancer is treated with a combination of a purinosome disrupting inhibitor, a prodrug therefor, or a pharmaceutically acceptable salt thereof; with a protein kinase inhibitor. The inhibited protein kinase can be a tyrosine kinase, a serine kinase, or a threonine kinase. The tyrosine kinase inhibitor can be a receptor tyrosine kinase inhibitor and can be selected from the group consisting of the EGFR and/or HER2 inhibitor erlotinib and lapatinib, the Src inhibitor dasatinib, the mTOR inhibitor temsirolimus and rapamycin, the Raf, VEGFR, PDGFR and c-kit inhibitor sorafenib, the PDGFR, c-kit and Flt3 inhibitor sunitinib, as well as enantiomers, prodrugs and pharmaceutically acceptable salts of those compounds. The combination of inhibitors of kinases and purinosome disrupting inhibitors can be used to treat many different cancers, including, in preferred embodiments, gastrointestinal stromal tumor, non-small-cell lung cancer, squamous cell carcinoma of the head and neck, and hormone refractory prostate cancer.

This invention provides new effective methods, compositions, and kits for treatment and/or prevention of various types of cancer. As used herein, cancer includes solid tumors and hematological malignancies. The solid tumors include cancers such as breast, colon, and ovarian cancers. The hematological malignancies include hematopoietic malignancies including leukemias, lymphomas and myelomas that are conditions characterized by abnormal growth and maturation of hematopoietic cells.

Leukemias are generally neoplastic disorders of hematopoietic stem cells, and include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Other hematological malignancies include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

Hematological malignancies are generally serious disorders, resulting in a variety of symptoms, including bone marrow failure and organ failure. Treatment for many hematological malignancies, including leukemias and lymphomas, remains difficult, and existing therapies are not universally effective. While treatments involving specific immunotherapy appear to have considerable potential, such treatments have been limited by the small number of known malignancy-associated antigens. Moreover the ability to detect such hematological malignancies in their early stages can be quite difficult depending upon the particular malady. Accordingly, there remains a need in the art for improved methods for treatment of hematological malignancies such as B cell leukemias and lymphomas and multiple myelomas. The present invention fulfills these and other needs in the field.

Other cancers include those characterized by solid tumors, including skin cancers (e.g., melanomas, basal cell carcinomas, and squamous cell carcinomas), epithelial carcinomas of the head and neck, lung cancer (e.g., squamous or epidermoid carcinoma, small cell carcinoma, adenocarcinoma, and large cell carcinoma), breast cancer (e.g., ductal carcinoma in situ and lobular neoplasia), gastrointestinal tract cancers (e.g., esophageal cancers, gastric adenocarcinoma, primary gastric lymphoma, colorectal cancer, small bowel tumors and cancers of the anus), pancreatic cancer, hepatocellular cancer, bladder cancer, renal cell carcinoma, pro static carcinoma, testicular cancer, ovarian cancer, carcinoma of the fallopian tube, uterine cancer, and cervical cancer, thyroid cancer (e.g., papillary, follicular, and anaplastic carcinomas).

Treatment of sarcomas of the bone and soft tissue are encompassed by the present invention. Bone sarcomas include osteosarcoma, chondrosarcoma, and Ewing's sarcoma.

In addition, slow growing cancers can include the following: chronic lymphocytic leukemia (CLL), chronic myelogenous leukemia, non-Hodgkins lymphoma, multiple myeloma, chronic granulocytic leukemia, cutaneous T cell lymphoma, low grade lymphomas, colon cancer, uterine cancer, breast cancer, prostate cancer, and thyroid cancer.

A major class of cancer treatment targets are the G protein coupled receptors (GPCRs). Many GPCR genes are selectively expressed by cancers. GPCRs share a common structure: seven membrane spanning domains, an extracellular domain and an intracellular domain. After ligand binding, the conformation of the GPCR changes and the intracellular domain activates a specific G-protein, either directly or through activation of a guanine nucleotide exchange factor (GEF), which activates another G-protein. In some cancers the expression of GPCRs is increased, leading to increased activation of G-proteins and their associated signal transduction pathways. In addition, some G-proteins downstream of GPCRs are oncogenes, and cancers with increased activity of downstream G-proteins can also be effectively treated by the combination of purinosome disrupting molecules and protein kinase inhibitors. One example of an oncogene downstream of a GPCR is the Rho oncogene.

a) Therapeutic Agents

As used herein, the term “therapeutic agent” means a molecule which can have one or more biological activities in a normal or pathologic tissue. The therapeutic agent can comprise a compound or composition for treating cancer. The therapeutic agent can comprise a compound or composition to induce programmed cell death or apoptosis. Membrane perturbing molecules are a form of therapeutic agent.

In some embodiments, the therapeutic agent can be a cancer chemotherapeutic agent. As used herein, a “cancer chemotherapeutic agent” is a chemical agent that inhibits the proliferation, growth, life-span or metastatic activity of cancer cells. Such a cancer chemotherapeutic agent can be, without limitation, a taxane such as docetaxel; an anthracyclin such as doxorubicin; an alkylating agent; a vinca alkaloid; an anti-metabolite; a platinum agent such as cisplatin or carboplatin; a steroid such as methotrexate; an antibiotic such as adriamycin; a isofamide; or a selective estrogen receptor modulator; an antibody such as trastuzumab.

Taxanes are chemotherapeutic agents useful with the compositions disclosed herein. Useful taxanes include, without limitation, docetaxel (Taxotere; Aventis Pharmaceuticals, Inc.; Parsippany, N.J.) and paclitaxel (Taxol; Bristol-Myers Squibb; Princeton, N.J.). See, for example, Chan et al., J. Clin. Oncol. 17:2341-2354 (1999), and Paridaens et al., J. Clin. Oncol. 18:724 (2000).

A cancer chemotherapeutic agent useful with the compositions disclosed herein also can be an anthracyclin such as doxorubicin, idarubicin or daunorubicin. Doxorubicin is a commonly used cancer chemotherapeutic agent and can be useful, for example, for treating breast cancer (Stewart and Ratain, In: “Cancer: Principles and practice of oncology” 5th ed., chap. 19 (eds. DeVita, Jr., et al.; J. P. Lippincott 1997); Harris et al., In “Cancer: Principles and practice of oncology,” supra, 1997). In addition, doxorubicin has anti-angiogenic activity (Folkman, Nature Biotechnology 15:510 (1997); Steiner, In “Angiogenesis: Key principles-Science, technology and medicine,” pp. 449-454 (eds. Steiner et al.; Birkhauser Verlag, 1992)), which can contribute to its effectiveness in treating cancer.

An alkylating agent such as melphalan or chlorambucil also can be a useful cancer chemotherapeutic agent. Similarly, a vinca alkaloid such as vindesine, vinblastine or vinorelbine; or an antimetabolite such as 5-fluorouracil, 5-fluorouridine or a derivative thereof can be a useful cancer chemotherapeutic agent.

A platinum agent also can be a useful cancer chemotherapeutic agent. Such a platinum agent can be, for example, cisplatin or carboplatin as described, for example, in Crown, Seminars in Oncol. 28:28-37 (2001). Other useful cancer chemotherapeutic agents include, without limitation, methotrexate, mitomycin-C, adriamycin, ifosfamide and ansamycins.

A cancer chemotherapeutic agent useful for treatment of breast cancer and other hormonally-dependent cancers also can be an agent that antagonizes the effect of estrogen, such as a selective estrogen receptor modulator or an anti-estrogen. The selective estrogen receptor modulator, tamoxifen, is a cancer chemotherapeutic agent that can be used in a composition for treatment of breast cancer (Fisher et al., J. Natl. Cancer Instit. 90:1371-1388 (1998)).

The therapeutic agent can be an antibody such as a humanized monoclonal antibody. As an example, the anti-epidermal growth factor receptor 2 (HER2) antibody trastuzumab (Herceptin; Genentech, South San Francisco, Calif.) can be a therapeutic agent useful for treating HER2/neu overexpressing breast cancers (White et al., Annu. Rev. Med. 52:125-141 (2001)).

The therapeutic agent can be a kinase or kinase inhibitor. For example, the protein kinase inhibitors can competitively compete with ATP. One example is the protein kinase inhibitor erlotinib, lapatinib, dasatinib, temsirolimus, rapamycin, sorafenib, and sunitinib.

Useful therapeutic agents also can be a cytotoxic agent, which, as used herein, can be any molecule that directly or indirectly promotes cell death. Useful cytotoxic agents include, without limitation, small molecules, polypeptides, peptides, peptidomimetics, nucleic acid-molecules, cells and viruses. As non-limiting examples, useful cytotoxic agents include cytotoxic small molecules such as doxorubicin, docetaxel or trastuzumab; antimicrobial peptides such as those described further below; pro-apoptotic polypeptides such as caspases and toxins, for example, caspase-8; diphtheria toxin A chain, Pseudomonas exotoxin A, cholera toxin, ligand fusion toxins such as DAB389EGF, ricinus communis toxin (ricin); and cytotoxic cells such as cytotoxic T cells. See, for example, Martin et al., Cancer Res. 60:3218-3224 (2000); Kreitman and Pastan, Blood 90:252-259 (1997); Allam et al., Cancer Res. 57:2615-2618 (1997); and Osborne and Coronado-Heinsohn, Cancer J. Sci. Am. 2:175 (1996). One skilled in the art understands that these and additional cytotoxic agents described herein or known in the art can be useful in the disclosed compositions and methods.

In one embodiment, a therapeutic agent can be a therapeutic polypeptide. As used herein, a therapeutic polypeptide can be any polypeptide with a biologically useful function. Useful therapeutic polypeptides encompass, without limitation, cytokines, antibodies, cytotoxic polypeptides; pro-apoptotic polypeptides; and anti-angiogenic polypeptides. As non-limiting examples, useful therapeutic polypeptides can be a cytokine such as tumor necrosis factor-α (TNF-α), tumor necrosis factor-β (TNF-β), granulocyte macrophage colony stimulating factor (GM-CSF), granulocyte colony stimulating factor (G-CSF), interferon-alpha (IFN-α), interferon-γ (IFN-γ), interleukin-1 (IL-1), interleukin-2 (IL-2), interleukin-3 (IL-3), interleukin-4 (IL-4), interleukin-6 (IL-6), interleukin-7 (IL-7), interleukin-10 (IL-10), interleukin-12 (IL-12), lymphotactin (LTN) or dendritic cell chemokine 1 (DC-CK1); an anti-HER2 antibody or fragment thereof; a cytotoxic polypeptide including a toxin or caspase, for example, diphtheria toxin A chain, Pseudomonas exotoxin A, cholera toxin, a ligand fusion toxin such as DAB389EGF or ricin; or an anti-angiogenic polypeptide such as angiostatin, endostatin, thrombospondin, platelet factor 4; anastellin; or one of those described further herein or known in the art. It is understood that these and other polypeptides with biological activity can be a “therapeutic polypeptide.”

A therapeutic agent can also be an anti-angiogenic agent. As used herein, the term “anti-angiogenic agent” means a molecule that reduces or prevents angiogenesis, which is the growth and development of blood vessels. A variety of anti-angiogenic agents can be prepared by routine methods. Such anti-angiogenic agents include, without limitation, small molecules; proteins such as dominant negative forms of angiogenic factors, transcription factors and antibodies; peptides; and nucleic acid molecules including ribozymes, antisense oligonucleotides, and nucleic acid molecules encoding, for example, dominant negative forms of angiogenic factors and receptors, transcription factors, and antibodies and antigen-binding fragments thereof. See, for example, Hagedorn and Bikfalvi, Crit. Rev. Oncol. Hematol. 34:89-110 (2000), and Kirsch et al., J. Neurooncol. 50:149-163 (2000).

Vascular endothelial growth factor (VEGF) has been shown to be important for angiogenesis in many types of cancer, including breast cancer angiogenesis in vivo (Borgstrom et al., Anticancer Res. 19:4213-4214 (1999)). The biological effects of VEGF include stimulation of endothelial cell proliferation, survival, migration and tube formation, and regulation of vascular permeability. An anti-angiogenic agent can be, for example, an inhibitor or neutralizing antibody that reduces the expression or signaling of VEGF or another angiogenic factor, for example, an anti-VEGF neutralizing monoclonal antibody (Borgstrom et al., supra, 1999). An anti-angiogenic agent also can inhibit another angiogenic factor such as a member of the fibroblast growth factor family such as FGF-1 (acidic), FGF-2 (basic), FGF-4 or FGF-5 (Slavin et al., Cell Biol. Int. 19:431-444 (1995); Folkman and Shing, J. Biol. Chem. 267:10931-10934 (1992)) or an angiogenic factor such as angiopoietin-1, a factor that signals through the endothelial cell-specific Tie2 receptor tyrosine kinase (Davis et al., Cell 87:1161-1169 (1996); and Sufi et al., Cell 87:1171-1180 (1996)), or the receptor of one of these angiogenic factors. It is understood that a variety of mechanisms can act to inhibit activity of an angiogenic factor including, without limitation, direct inhibition of receptor binding, indirect inhibition by reducing secretion of the angiogenic factor into the extracellular space, or inhibition of expression, function or signaling of the angiogenic factor.

A variety of other molecules also can function as anti-angiogenic agents including, without limitation, angiostatin; a kringle peptide of angiostatin; endostatin; anastellin, heparin-binding fragments of fibronectin; modified forms of antithrombin; collagenase inhibitors; basement membrane turnover inhibitors; angiostatic steroids; platelet factor 4 and fragments and peptides thereof; thrombospondin and fragments and peptides thereof; and doxorubicin (O'Reilly et al., Cell 79:315-328 (1994)); O'Reilly et al., Cell 88:277-285 (1997); Homandberg et al., Am. J. Path. 120:327-332 (1985); Homandberg et-al., Biochim. Biophys. Acta 874:61-71 (1986); and O'Reilly et al., Science 285:1926-1928 (1999)). Commercially available anti-angiogenic agents include, for example, angiostatin, endostatin, metastatin and 2ME2 (EntreMed; Rockville, Md.); anti-VEGF antibodies such as Avastin (Genentech; South San Francisco, Calif.); and VEGFR-2 inhibitors such as SU5416, a small molecule inhibitor of VEGFR-2 (SUGEN; South San Francisco, Calif.) and SU6668 (SUGEN), a small molecule inhibitor of VEGFR-2, platelet derived growth factor and fibroblast growth factor I receptor. It is understood that these and other anti-angiogenic agents can be prepared by routine methods and are encompassed by the term “anti-angiogenic agent” as used herein.

Examples of useful therapeutic agents include but are not limited to steroids, fibronectin, anti-clotting drugs, anti-platelet function drugs, drugs which prevent smooth muscle cell growth on inner surface wall of vessel, heparin, heparin fragments, aspirin, coumadin, tissue plasminogen activator (TPA), urokinase, hirudin, streptokinase, antiproliferatives (methotrexate, cisplatin, fluorouracil, Adriamycin), antioxidants (ascorbic acid, beta carotene, vitamin E), antimetabolites, thromboxane inhibitors, non-steroidal and steroidal anti-inflammatory drugs, beta and calcium channel blockers, genetic materials including DNA and RNA fragments, complete expression genes, antibodies, lymphokines, growth factors, prostaglandins, leukotrienes, laminin, elastin, collagen, and integrins.

Useful therapeutic agents also can be antimicrobial peptides. Thus, for example, also disclosed are therapeutic agents comprising an antimicrobial peptide, where the composition is selectively internalized and exhibits a high toxicity to the targeted area. Useful antimicrobial peptides can have low mammalian cell toxicity when not incorporated into the composition. As used herein, the term “antimicrobial peptide” means a naturally occurring or synthetic peptide having antimicrobial activity, which is the ability to kill or slow the growth of one or more microbes. An antimicrobial peptide can, for example, kill or slow the growth of one or more strains of bacteria including a Gram-positive or Gram-negative bacteria, or a fungi or protozoa. Thus, an antimicrobial peptide can have, for example, bacteriostatic or bacteriocidal activity against, for example, one or more strains of Escherichia coli, Pseudomonas aeruginosa or Staphylococcus aureus. While not wishing to be bound by the following, an antimicrobial peptide can have biological activity due to the ability to form ion channels through membrane bilayers as a consequence of self-aggregation.

An antimicrobial peptide is typically highly basic and can have a linear or cyclic structure. As discussed further below, an antimicrobial peptide can have an amphipathic .alpha.-helical structure (see U.S. Pat. No. 5,789,542; Javadpour et al., J. Med. Chem. 39:3107-3113 (1996); and Blondelle and Houghten, Biochem. 31: 12688-12694 (1992)). An antimicrobial peptide also can be, for example, a β-strand/sheet-forming peptide as described in Mancheno et al., J. Peptide Res. 51:142-148 (1998).

An antimicrobial peptide can be a naturally occurring or synthetic peptide. Naturally occurring antimicrobial peptides have been isolated from biological sources such as bacteria, insects, amphibians, and mammals and are thought to represent inducible defense proteins that can protect the host organism from bacterial infection. Naturally occurring antimicrobial peptides include the gramicidins, magainins, mellitins, defensins and cecropins (see, for example, Maloy and Kari, Biopolymers 37:105-122 (1995); Alvarez-Bravo et al., Biochem. J. 302:535-538 (1994); Bessalle et al., FEBS 274:-151-155 (1990); and Blondelle and Houghten in Bristol (Ed.), Annual Reports in Medicinal Chemistry pages 159-168 Academic Press, San Diego). An antimicrobial peptide also can be an analog of a natural peptide, especially one that retains or enhances amphipathicity.

An antimicrobial peptide incorporated into the composition disclosed herein can have low mammalian cell toxicity when linked to the composition. Mammalian cell toxicity readily can be assessed using routine assays. As an example, mammalian cell toxicity can be assayed by lysis of human erythrocytes in vitro as described in Javadpour et al., supra, 1996. An antimicrobial peptide having low mammalian cell toxicity is not lytic to human erythrocytes or requires concentrations of greater than 100 μM for lytic activity, preferably concentrations greater than 200, 300, 500 or 1000 μM.

In one embodiment, disclosed are compositions in which the antimicrobial peptide portion promotes disruption of mitochondrial membranes when internalized by eukaryotic cells. In particular, such an antimicrobial peptide preferentially disrupts mitochondrial membranes as compared to eukaryotic membranes. Mitochondrial membranes, like bacterial membranes but in contrast to eukaryotic plasma membranes, have a high content of negatively charged phospholipids. An antimicrobial peptide can be assayed for activity in disrupting mitochondrial membranes using, for example, an assay for mitochondrial swelling or another assay well known in the art.

An antimicrobial peptide that induces significant mitochondrial swelling at, for example, 50 μM, 40 μ.M, 30 μM, 20 μM, 10 μM, or less, is considered a peptide that promotes disruption of mitochondrial membranes.

Antimicrobial peptides generally have random coil conformations in dilute aqueous solutions, yet high levels of helicity can be induced by helix-promoting solvents and amphipathic media such as micelles, synthetic bilayers or cell membranes. α-Helical structures are well known in the art, with an ideal α-helix characterized by having 3.6 residues per turn and a translation of 1.5 Å per residue (5.4 Å per turn; see Creighton, Proteins: Structures and Molecular Properties W. H Freeman, New York (1984)). In an amphipathic α-helical structure, polar and non-polar amino acid residues are aligned into an amphipathic helix, which is an α-helix in which the hydrophobic amino acid residues are predominantly on one face, with hydrophilic residues predominantly on the opposite face when the peptide is viewed along the helical axis.

Antimicrobial peptides of widely varying sequence have been isolated, sharing an amphipathic α-helical structure as a common feature (Saberwal et al., Biochim. Biophys. Acta 1197:109-131 (1994)). Analogs of native peptides with amino acid substitutions predicted to enhance amphipathicity and helicity typically have increased antimicrobial activity. In general, analogs with increased antimicrobial activity also have increased cytotoxicity against mammalian cells (Maloy et al., Biopolymers 37:105-122 (1995)).

As used herein in reference to an antimicrobial peptide, the term “amphipathic α-helical structure” means an α-helix with a hydrophilic face containing several polar residues at physiological pH and a hydrophobic face containing nonpolar residues. A polar residue can be, for example, a lysine or arginine residue, while a nonpolar residue can be, for example, a leucine or alanine residue. An antimicrobial peptide having an amphipathic .alpha.-helical structure generally has an equivalent number of polar and nonpolar residues within the amphipathic domain and a sufficient number of basic residues to give the peptide an overall positive charge at neutral pH (Saberwal et al., Biochim. Biophys. Acta 1197:109-131 (1994)). One skilled in the art understands that helix-promoting amino acids such as leucine and alanine can be advantageously included in an antimicrobial peptide (see, for example, Creighton, supra, 1984). Synthetic, antimicrobial peptides having an amphipathic α-helical structure are known in the art, for example, as described in U.S. Pat. No. 5,789,542 to McLaughlin and Becker.

It is understood by one skilled in the art of medicinal oncology that these and other agents are useful therapeutic agents, which can be used separately or together in the disclosed compositions and methods. Thus, it is understood that the compositions disclosed herein can contain one or more of such therapeutic agents and that additional components can be included as part of the composition, if desired. As a non-limiting example, it can be desirable in some cases to utilize an oligopeptide spacer between the surface molecule and the homing molecule and/or cargo molecules (Fitzpatrick and Garnett, Anticancer Drug Des. 10:1-9 (1995)).

Other useful agents include thrombolytics, aspirin, anticoagulants, painkillers and tranquilizers, beta-blockers, ace-inhibitors, nitrates, rhythm-stabilizing drugs, and diuretics. Agents that limit damage to the heart work best if given within a few hours of the heart attack. Thrombolytic agents that break up blood clots and enable oxygen-rich blood to flow through the blocked artery increase the patient's chance of survival if given as soon as possible after the heart attack. Thrombolytics given within a few hours after a heart attack are the most effective. Injected intravenously, these include anisoylated plasminogen streptokinase activator complex (APSAC) or anistreplase, recombinant tissue-type plasminogen activator (r-tPA), and streptokinase. The disclosed compounds can use any of these or similar agents.

Some other examples of useful therapeutic agents include nitrogen mustards, nitrosoureas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents. Exemplary chemotherapy drugs are Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Chlomaphazine, Cholophosphamide, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Estramustine, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, mechlorethamine oxide hydrochloride, Melphalan, Mercaptopurine, Methotrexate, Mitomycin, Mitotane, Mitoxantrone, Novembiehin, Oxaliplatin, Paclitaxel, Pamidronate, Pentostatin, Phenesterine, Plicamycin, Prednimustine, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Trofosfamide, Vinblastine, Vincristine, Vindesine, Vinorelbine, VP-16, and Xeloda. Alkylating agents such as Thiotepa and; alkyl sulfonates such as Busulfan, Improsulfan and Piposulfan; aziridines such as Benzodopa, Carboquone, Meturedopa, and Uredopa; ethylenimines and methylamelamines including altretamine, triethylenemelamine, trietylenephosphoramide, triethylenethiophosphaoramide and trimethylolomelamine; nitroureas such as Cannustine, Chlorozotocin, Fotemustine, Lomustine, Nimustine, and Ranimustine; antibiotics such as Aclacinomysins, Actinomycin, Authramycin, Azaserine, Bleomycins, Cactinomycin, Calicheamicin, Carabicin, Caminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites such as Methotrexate and 5-fluorouracil (5-FU); folic acid analogues such as Denopterin, Methotrexate, Pteropterin, and Trimetrexate; purine analogs such as Fludarabine, 6-mercaptopurine, Thiamiprine, and Thioguanine; pyrimidine analogs such as Ancitabine, Azacitidine, 6-azauridine, Carmofur, Cytarabine, Dideoxyuridine, Doxifluridine, Enocitabine, Floxuridine, and 5-FU; androgens such as Calusterone, Dromostanolone Propionate, Epitiostanol, Rnepitiostane, and Testolactone; anti-adrenals such as aminoglutethimide, Mitotane, and Trilostane; folic acid replenisher such as frolinic acid; aceglatone; aldophosphamide glycoside; aminolevulinic acid; Amsacrine; Bestrabucil; Bisantrene; Edatraxate; Defofamine; Demecolcine; Diaziquone; Elformithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2,2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (TAXOL®, Bristol-Myers Squibb Oncology, Princeton, N.J.) and Doxetaxel (TAXOTERE®, Rhone-Poulenc Rorer, Antony, France); Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylornithine (DMFO); retinoic acid; Esperamicins; Capecitabine; and pharmaceutically acceptable salts, acids or derivatives of any of the above. Also included are anti-hormonal agents that act to regulate or inhibit hormone action on tumors such as anti-estrogens including for example Tamoxifen, Raloxifene, aromatase inhibiting 4(5)-imidazoles, 4 Hydroxytamoxifen, Trioxifene, Keoxifene, Onapristone, And Toremifene (Fareston); and anti-androgens such as Flutamide, Nilutamide, Bicalutamide, Leuprolide, and Goserelin; and pharmaceutically acceptable salts, acids or derivatives of any of the above. Useful cargo molecules include, for example, doxorubicin, Herceptin, and liposomal doxorubicin.

The cargo molecules can also comprise a boron containing compound. Boron containing compounds have received increasing attention as therapeutic agents over the past few years as technology in organic synthesis has expanded to include this atom (Boron Therapeutics on the horizon, Groziak, M. P.; American Journal of Therapeutics (2001) 8, 321-328). The most notable boron containing therapeutic is the boronic acid bortezomib which was recently launched for the treatment of multiple myeloma. This breakthrough demonstrates the feasibility of using boron containing compounds as pharmaceutical agents. Boron containing compounds have been shown to have various biological activities including herbicides (Organic boron compounds as herbicides. Barnsley, G. E.; Eaton, J. K.; Airs, R. S.; (1957), DE 1016978 19571003), boron neutron capture therapy (Molecular Design and Synthesis of B-10 Carriers for Neutron Capture Therapy. Yamamoto, Y.; Pure Appl. Chem., (1991) 63, 423-426), serine protease inhibition (Borinic acid inhibitors as probes of the factors involved in binding at the active sites of subtilisin Carlsberg and .alpha.-chymotrypsin. Simpelkamp, J.; Jones, J. B.; Bioorganic & Medicinal Chemistry Letters, (1992), 2 (11), 1391-4; Design, Synthesis and Biological Evaluation of Selective Boron-containing Thrombin Inhibitors. Weinand, A.; Ehrhardt, C.; Metternich, R.; Tapparelli, C.; Bioorganic and Medicinal Chemistry, (1999), 7, 1295-1307), acetylcholinesterase inhibition (New, specific and reversible bifunctional alkylborinic acid inhibitor of acetylcholinesterase. Koehler, K. A.; Hess, G. P.; Biochemistry (1974), 13, 5345-50) and as antibacterial agents (Boron-Containing Antibacterial Agents: Effects on Growth and Morphology of Bacteria Under Various Culture Conditions. Bailey, P. J.; Cousins, G.; Snow, G. A.; and White, A. J.; Antimicrobial Agents and Chemotherapy, (1980), 17, 549-553). The boron containing compounds with antibacterial activity can be sub-divided into two main classes, the diazaborinines, which have been known since the 1960's, and dithienylborinic acid complexes. This latter class has been expanded to include many different diarylborinic acid complexes with potent antibacterial activity (Preparation of diarylborinic acid esters as DNA methyl transferase inhibitors. Benkovic, S. J.; Shapiro, L.; Baker, S. J.; Wahnon, D. C.; Wall, M.; Shier, V. K.; Scott, C. P.; Baboval, J.; PCT Int. Appl. (2002), WO 2002044184).

### C. Materials

Purinosome promoting CK2 inhibitors include, but are not limited to, DMAT, TBCA and DRB. Purinosome disrupting CK2 inhibitors include, but are not limited to, TBB and TBBz. Purinosome promoting agents in general also include any molecules that promote the purinosome formation. For example, the alpha2A adrenergic receptor agonists can be used as a purinosome promoting agent in Hela cells.

Disclosed are purinosome dynamics modulators and pharmaceutically acceptable salts thereof. The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds of the present invention contain both basic and acidic functionalities that allow the compounds to be converted into either base or acid addition salts.

In addition to salt forms, the present invention contemplates compounds that are in a prodrug form. Prodrugs of the compounds described herein are those compounds that readily undergo chemical changes under physiological conditions to provide compounds having the inhibitory activity desired within the present invention. Thus, prodrugs can undergo more than one chemical change under physiological conditions to provide an inhibitory activity. Additionally, prodrugs can be converted to compounds having the desired inhibitory activity by chemical or biochemical methods in an ex vivo environment. For example, prodrugs can be slowly converted to compounds having the desired inhibitory activity within the present invention when placed in a transdermal patch reservoir with a suitable enzyme or chemical reagent.

1. Biosensors

Label-free cell-based assays generally employ a biosensor to monitor molecule-induced responses in living cells. The molecule can be naturally occurring or synthetic, and can be a purified or unpurified mixture. A biosensor typically utilizes a transducer such as an optical, electrical, calorimetric, acoustic, magnetic, or like transducer, to convert a molecular recognition event or a molecule-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present methods can include, for example, optical biosensor systems such as surface plasmon resonance (SPR) and resonant waveguide grating (RWG) biosensors, photonic crystal biosensors, resonant mirrors, ellipsometers, and electric biosensor systems such as bioimpedance systems.

RWG biosensor belongs to a family of label-free optical biosensors that is sensitive to alterations in local refractive index at or near the sensor surface. A RWG biosensor including photonic crystal biosensor consists of three components: a biological component, a detector element, and a transducer associated with both components.

The biological component is a live cell or a tissue cell for whole cell sensing. For an anchorage dependent cell, the cell can be directly cultured onto the transducer surface to form an adherent layer of cells. For suspension cells (e.g., lymphoblastic leukemia cells), they can be brought to closely contact with the transducer surface via physical sedimentation or specific biochemical binding between the immobilized molecule and a cell surface molecule.

The RWG transducer is a nano-grating waveguide. For live cell sensing, the biosensor is often considered as a three-layer waveguide configuration: a substrate with a diffractive grating, a high index of refraction waveguide coating, and a cell layer. Such a configuration supports both transverse magnetic (TM₀) and transverse electric (TE₀) modes. The TM₀ mode has higher sensitivity and longer penetration depth (i.e., larger sensing volume for live cells) but relatively lower spatial resolution (˜tens of micrometers), comparing to the TE₀ mode. Thus, most RWG biosensors use the TM₀ mode for whole cell sensing. The penetration depth is the distance from the sensor surface at which the electric field strength has decreased to 1/e of its initial value (typically ˜150 nm). The electromagnetic field, termed evanescent wave whose intensity exponentially decays away from the sensor surface, is created by the diffraction grating coupled waveguide resonance. This indicates that the biosensor only samples the bottom portion of the cells contacting with the sensor surface.

The RWG detector exploits resonant coupling of light into a waveguide via the diffraction grating. When illuminated with broadband light at a fixed and nominally normal angle of incidence, these sensors reflect only a narrow band of wavelengths (resonant wavelength) that is a sensitive function of the local index of refraction of the biosensor. Since the local index of refraction is directly proportional to the density and distribution of biomass (e.g., proteins, molecular complexes) in live cells, the RWG can non-invasively detect stimulus-induced DMR in native cells. The DMR defines redistribution of cellular matters within the sensing volume. Such redistribution is often not random; instead, it is tightly regulated and is often dynamic both spatially and temporally. The biosensor simply acts a non-invasive monitor to record the DMR in real time. In general, a DMR signal may contain contributions from protein trafficking, microfilament remodeling, and cell adhesion alterations. However, different events may dominate different DMR signals. Furthermore, multiple parameters can be derived from a DMR signal, and used for characterizing receptor signaling and drug pharmacology. Therefore, it is not surprising to see that in recent years RWG biosensor cellular assays have found broad applications to a diverse array of cellular processes, including adhesion, cell viral infection, proliferation and apoptosis of cells. These assays are also amenable to a wide range of receptors, including G protein-coupled receptors (GPCRs), ion channels, kinases, enzymes, and structural proteins. Numerous studies have found that the DMR measurements are pathway-sensitive, and often reflect the complexity of receptor biology and drug pharmacology. More importantly, the DMR measurements consist of contributions from many cellular events downstream the receptor of interest, enabling the identification of many critical nodes and core pathways in receptor signalling network.

RWG biosensor systems are commercially available nowadays. The Epic® system (Corning Inc) is the first optical biosensor that is amenable to microtiter plate-based high throughput screening (HTS) for both biomolecular interaction analysis and cell-based assays. The system consists of a RWG detector, an external liquid handling accessory and a scheduler, such that it can process large numbers of microplates using end-point measurements for HTS, or using kinetic measurements for high information content screening. The detector utilizes a linear array of fiber optics to rapidly scan a whole microtiter plate, and to track changes in the central wavelength (resonant wavelength) of the biosensor resonant spectrum. Epic® biosensor microplates (typically 384 well format) have appropriate surface coatings for different applications.

2. Pharmaceutical Compositions

Disclosed are pharmaceutical compositions for treating a subject comprising a therapeutically effective amount of a purinosome dynamics modulating molecule.

In some forms, the pharmaceutical compositions further comprise one or more therapeutic agents. The therapeutic agent can be an anti-cancer agent, anti-inflammatory agent, or anti-angiogenic agent.

In some forms of the pharmaceutical compositions, the purinosome dynamics modulating molecule and the therapeutic agent produce a synergistic effect in treating diseases which are pathophysiologically related to purinosomes.

In some forms of the pharmaceutical compositions, the subject can be a mammal

In some forms, the pharmaceutical compositions as described above can further comprise a pharmaceutically acceptable carrier or excipient. By “pharmaceutically acceptable”, it is meant a material that is not biologically or otherwise undesirable, i.e., the material can be administered to a subject without causing any undesirable biological effects or interacting in a deleterious manner with any of the other components of the pharmaceutical composition in which it is contained. A pharmaceutically acceptable component is one that is suitable for use with humans and/or animals without undue adverse side effects (such as toxicity, irritation, and allergic response) commensurate with a reasonable benefit/risk ratio.

The carrier can be selected to minimize any degradation of the active ingredient and to minimize any adverse side effects in the subject, as would be well known to one of skill in the art. The carrier can be a solid, a liquid, or both, and can be formulated with the compound as a unit-dose composition, for example, a tablet, which can contain, for example, from 0.05% to 100%, from 0.05% to 99%, from 0.05% to 98%, from 0.05% to 97%, from 0.05% to 96%, from 0.05% to 95%, from 0.05% to 94%, from 0.05% to 93%, from 0.05% to 92%, from 0.05% to 91%, from 0.05% to 90%, from 0.05% to 85%, from 0.05% to 80%, from 0.05% to 75%, from 0.05% to 70%, from 0.05% to 65%, from 0.05% to 60%, from 0.05% to 55%, from 0.05% to 50%, from 0.05% to 45%, from 0.05% to 40%, from 0.05% to 35%, from 0.05% to 30%, from 0.05% to 25%, from 0.05% to 20%, from 0.05% to 15%, from 0.05% to 10%, from 0.05% to 5%, from 0.05% to 4%, from 0.05% to 3%, from 0.05% to 2%, from 0.05% to 1%, from 0.05% to 0.8%, from 0.05% to 0.6%, from 0.05% to 0.5%, from 0.05% to 0.4%, from 0.05% to 0.3%, from 0.05% to 0.2%, from 0.05% to 0.1%, from 0.1% to 100%, from 0.2% to 100%, from 0.3% to 100%, from 0.4% to 100%, from 0.5% to 100%, from 0.6% to 100%, from 0.8% to 100%, from 1% to 100%, from 2% to 100%, from 3% to 100%, from 4% to 100%, from 5% to 100%, from 10% to 100%, from 15% to 100%, from 20% to 100%, from 25% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.8%, 0.6%, 0.5%, 0.4%, 0.3%, 0.2%, 0.1%, 0.05% by weight of the active compounds. A disclosed compound can be coupled with suitable polymers as targetable drug carriers. Other pharmacologically active substances can also be present.

Any suitable route of administration can be used for the disclosed compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, vaginal, intravaginal, transvaginal, ocular, intraocular, transocular, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc.

Suitable carriers and their formulations are described in Remington: The Science and Practice of Pharmacy (19th ed.) ed. A. R. Gennaro, Mack Publishing Company, Easton, Pa., 1995. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing, for example, the antiviral agent, which matrices can be in the form of shaped articles, e.g., films, liposomes or microparticles. It will be apparent to those persons skilled in the art that certain carriers may be more preferable depending upon, for instance, the route of administration and concentration of composition being administered. Pharmaceutical carriers are known to those skilled in the art. These most typically would be standard carriers for administration of drugs to humans, including solutions such as sterile water, saline, and buffered solutions at physiological pH. Examples of the pharmaceutically acceptable excipient include, but are not limited to, thickeners, diluents, buffers, preservatives, surface active agents and the like.

The disclosed compounds can be administered by any suitable route, preferably in the form of a pharmaceutical composition adapted to such a route, and in a dose effective for the treatment or prevention intended. The active compounds and compositions, for example, can be administered orally, rectally, parenterally, ocularly, inhalationaly, or topically.

Oral administration of a solid dose form can be, for example, presented in discrete units, such as hard or soft capsules, pills, cachets, lozenges, or tablets, each containing a predetermined amount of at least one of the disclosed compound or compositions. In some forms, the oral administration can be in a powder or granule form. In some forms, the oral dose form is sub-lingual, such as, for example, a lozenge. In such solid dosage forms, the compounds of formula I are ordinarily combined with one or more adjuvants. Such capsules or tablets can contain a controlled-release formulation. In the case of capsules, tablets, and pills, the dosage forms also can comprise buffering agents or can be prepared with enteric coatings.

In some forms, oral administration can be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), and/or perfuming agents.

In some forms, the disclosed compositions can comprise a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) can be formulated according to the known art using suitable dispersing, wetting agents, and/or suspending agents.

In some forms, the disclosed compositions can comprise a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation can include a compound which enhances absorption or penetration of the active ingredient through the skin or other affected areas. When the compounds and compositions are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes can also be used. Typical carriers include alcohol, water, mineral oil, liquid petrolatum, white petrolatum, glycerin, polyethylene glycol and propylene glycol. Penetration enhancers can be incorporated—see, for example, J Pharm Sci, 88 (10), 955-958, by Finnin and Morgan (October 1999).

Formulations suitable for topical administration to the eye include, for example, eye drops wherein the disclosed compound or composition is dissolved or suspended in suitable carrier. A typical formulation suitable for ocular or aural administration can be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, can be incorporated together with a preservative, such as benzalkonium chloride. Such formulations can also be delivered by iontophoresis.

For intranasal administration or administration by inhalation, the active disclosed compounds are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder can comprise a bioadhesive agent, for example, chitosan or cyclodextrin.

In some forms, the disclosed compositions can comprise a rectal dose form. Such rectal dose form can be in the form of, for example, a suppository. Cocoa butter is a traditional suppository base, but various alternatives can be used as appropriate.

Other carrier materials and modes of administration known in the pharmaceutical art can also be used. The disclosed pharmaceutical compositions can be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The disclosed cellular targets, cellular pathway modulators or purinosome modulating molecules, alone or in combination with other therapeutic agents, in the treatment or prevention of various conditions or disease states. The disclosed molecule(s) and composition(s) and other therapeutic agent(s) can be administered simultaneously (either in the same dosage form or in separate dosage forms) or sequentially. An exemplary therapeutic agent can be, for example, one selected from the group consisting of anti-cancer agent, anti-inflammation agent, anti-metabolic-disorder agent, or anti-congestive-heart-failure agent.

The administration of two or more compounds “in combination” means that the two compounds are administered closely enough in time that the presence of one alters the biological effects of the other. The two or more compounds can be administered simultaneously, concurrently or sequentially. Additionally, simultaneous administration can be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

The dosage regimen for the compounds and/or compositions containing the compounds can be based on a variety of factors, including the type, age, weight, sex and medical condition of the patient; the severity of the condition; the route of administration; and the activity of the particular compound employed. Thus the dosage regimen can vary widely. Dosage levels of the order from about 0.001 mg to about 100 mg per kilogram of body weight per day are useful in the treatment or prevention of the above-indicated conditions. Other effective dosages regimens of a disclosed compounds (administered in single or divided doses) include but are not limited to: from about 0.01 to about 100 mg/kg/day, from about 0.1 to about 50 mg/kg/day, from about 0.5 to about 30 mg/kg/day, from about 0.01 to about 10 mg/kg/day, and from about 0.1 to about 1.0 mg/kg/day. Dosage unit compositions can contain such amounts or submultiples thereof to make up the daily dose. In many instances, the administration of the compound will be repeated a plurality of times in a day. Multiple doses per day typically can be used to increase the total daily dose, if desired.

For oral administration, the compositions can be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or from about 1 mg to about 100 mg of active ingredient. Intravenously, doses can range from about 0.1 to about 10 mg/kg/minute during a constant rate infusion.

Disclosed are pharmaceutical compositions comprising an effective amount of a compound of the invention or a pharmaceutically accepted salt, solvate, clathrate, or prodrug thereof; and a pharmaceutically acceptable carrier or vehicle. These compositions may further comprise additional agents. These compositions are useful for modulating the dynamics of purinosomes, thus they improve the prevention and treatment of purinosome associated human diseases such as cancer.

3. Kits

Also disclosed are kits that are suitable for use in performing the methods of treatment or prevention described below. In some forms, the kit contains a first dosage form comprising one or more of the disclosed compounds and a container for the dosage, in quantities sufficient to carry out the disclosed methods. In some forms, a kit can comprise one or more disclosed compounds, and one or more other therapeutic agents. An exemplary therapeutic agent can be, for example, an anti-cancer agent.

4. Mixtures

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

5. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

6. Data Structures and Computer Control

Disclosed are data structures used in, generated by, or generated from, the disclosed method. Data structures generally are any form of data, information, and/or objects collected, organized, stored, and/or embodied in a composition or medium. A purinosome modulating molecule structure or protocol stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### D. Uses

The disclosed methods and compositions are applicable to numerous areas including, but not limited to, use to identify purinosome dynamics modulating molecules, use in assays to identify purinosome modulating pathways, and use to treat cancer. Other uses are disclosed, apparent from the disclosure, and/or will be understood by those in the art.

The disclosed methods and compositions provide ways of treating cancer using purinosome disrupting inhibitors. The inhibitors of the purinosome, a multienzyme complex important for purine de novo synthesis pathway, can be used alone or combined with protein kinase inhibitors. Inhibitors of protein kinases include EGFR tyrosine kinase inhibitors. The present invention also provides a gatekeeper assay for assessing the therapeutic potentials of any molecules in cancer prevention and treatment.

### E. Definitions

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

5. Assaying the Response

“Assaying the response” or like terms means using a means to characterize the response. For example, if a molecule is brought into contact with a cell, a biosensor can be used to assay the response of the cell upon exposure to the molecule.

6. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

7. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

8. Cellular Pathway Modulator

A “cellular pathway modulator” is a molecule that intervenes a known cellular pathway. Examples are PTX as a Gi mediated signaling pathway modulator, cholera toxin as a Gs mediated signaling pathway modulator, PI3K inhibitor LY294002 as a PI3K pathway modulator.

9. Cellular Response

A “cellular response” or like terms is any reaction by the cell to a stimulation.

10. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

11. Cellular Target

A “cellular target” or like terms is a biopolymer such as a protein or nucleic acid whose activity can be modified by an external stimulus. Cellular targets are most commonly proteins such as enzymes, kinases, ion channels, and receptors.

12. Characterizing

Characterizing or like terms refers to gathering information about any property of a substance, such as a ligand, molecule, marker, or cell, such as obtaining a profile for the ligand, molecule, marker, or cell.

13. Comprise

Throughout the description and claims of this specification, the word “comprise” and variations of the word, such as “comprising” and “comprises,” means “including but not limited to,” and is not intended to exclude, for example, other additives, components, integers or steps.

14. Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

15. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

16. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e., such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

17. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

18. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

19. De Novo Pathway of Purine Biosynthesis

The phrase “de novo pathway of purine biosynthesis” refers to enzymatic synthesis of purine in a multi-step pathway beginning with the formation of phosphoribosyl pyrophosphate (PRPP) and continuing to the synthesis of inosine monophosphate (IMP). The de novo pathway of purine biosynthesis also includes synthesis of precursors or cofactors of the substituents of the pathway, e.g., folate, tetrahydrofolate and derivatives. The de novo pathway of purine biosynthesis also includes enzymatic reactions that synthesize AMP, GMP, and corresponding diphosphates and triphosphates.

20. Detect

Detect or like terms refer to an ability of the apparatus and methods of the disclosure to discover or sense a molecule- or a marker-induced cellular response and to distinguish the sensed responses for distinct molecules.

21. DMAT

DMAT is 2-Dimethylamino-4,5,6,7-tetrabromo-1H-benzimidazole.

22. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

23. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell. Inhibit or forms of inhibit or like terms refers to reducing or suppressing.

24. In the Presence of the Molecule

“in the presence of the molecule” or like terms refers to the contact or exposure of the cultured cell with the molecule. The contact or exposure can be taken place before, or at the time, the stimulus is brought to contact with the cell.

25. Known Molecule

A known molecule or like terms is a molecule with known pharmacological/biological/physiological/pathophysiological activity whose precise mode of action(s) may be known or unknown.

26. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

27. Library

A library or like terms is a collection. The library can be a collection of anything disclosed herein. For example, it can be a collection of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library. A library can be for example, random or non-random, determined or undetermined. For example, disclosed are libraries of DMR indexes or biosensor indexes of known modulators.

28. Material

Material is the tangible part of something (chemical, biochemical, biological, or mixed) that goes into the makeup of a physical object.

29. Mimic

As used herein, “mimic” or like terms refers to performing one or more of the functions of a reference object. For example, a molecule mimic performs one or more of the functions of a molecule.

30. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

31. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

32. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

33. Molecule Incubated Cell

A molecule incubated cell or like terms is a cell that has been exposed to a molecule.

34. Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

35. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

36. Or

The word “or” or like terms as used herein means any one member of a particular list and also includes any combination of members of that list.

37. Pathophysiologically Related to Purinosomes

Something is “pathophysiologically related to purinosomes” if purinosomes are involved in the functional changes in the body associated with or resulting from disease or injury.

38. Positive Control

A “positive control” or like terms is a control that shows that the conditions for data collection can lead to data collection.

39. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

40. Potency

Potency or like terms is a measure of molecule activity expressed in terms of the amount required to produce an effect of given intensity. For example, a highly potent drug evokes a larger response at low concentrations. The potency is proportional to affinity and efficacy Affinity is the ability of the drug molecule to bind to a receptor.

41. Protein Kinase

A “protein kinase” is an enzyme that phosphorylates an amino acid residue (specifically, certain serine, threonine, or tyrosine residues) on a protein. Thus, protein kinase encompasses serine protein kinases, threonine protein kinases, and tyrosine protein kinases.

42. Protein Kinase Inhibitor

A “protein kinase inhibitor” or “inhibitor of a protein kinase”, or like terms, are compounds or agents that reduce the activity of the enzyme. A protein kinase inhibitor can reduce the activity of an enzyme by binding to the enzyme. Thus, a protein kinase inhibitor can inhibit activity of the enzyme in a competitive, or a noncompetitive manner.

43. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

44. Purinosome

The term “purinosome” refers to the multienzyme complex involved in purine de novo biosynthesis.

45. Purinosome Modulating Agent

The term “purinosome modulating agent” or “purinosome modulator” refers to any agent that can modulate a purinosome complex. A purinosome modulating agent can modulate the formation or dissociation of the purinosome.

46. Purinosome Promoting Agent

A purinosome promoting agent is any molecule that increases or promotes the formation, function or activity of a purinosome relative to a control. The agent can increase or stabilize the formation of an already formed purinosome relative to a control.

47. Purinosome-Promoting CK2 Inhibitor

A purinosome promoting CK2 inhibitor is any CK2 inhibitor that causes or increases the formation of purinosome complexes relative to a control.

48. Purinosome Disrupting CK2 Inhibitor

A purinosome disrupting CK2 inhibitor is any CK2 inhibitor that causes or increases the dissociation of an already formed purinosome complexes, or inhibits, reduces, prevents or decreases the formation of purinosome complexes relative to a control.

49. Purinosome Dynamics

The term “purinosome dynamics” refers to the ability of the purinosome to be regulated. The purinosome can associate and dissociate and this process is reversible. For example, DMAT can promote purinosome formation and sequential administration of TBB can reverse the DMAT effects and cause purinosome disruption.

50. Purinosome Dynamics Modulator

A purinosome dynamics modulator is a molecule that can modulate the dynamics of purinosomes directly or indirectly.

51. Purinosome Dynamics Modulating Pathway

The phrase “purinosome dynamics modulating pathway” refers to any pathway involved in regulating purinosome dynamics For example, the alpha2A adrenergic receptor pathway involving Gᵢ is a purinosome dynamics modulating pathway in Hela cells.

52. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

53. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

54. Response

A response or like terms is any reaction to any stimulation.

55. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

56. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

57. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

58. Substance

A substance or like terms is any physical object. A material is a substance. Molecules, ligands, markers, cells, proteins, and DNA can be considered substances. A machine or an article would be considered to be made of substances, rather than considered a substance themselves.

59. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

60. Synergistic Effect

The term “synergistic effect” refers to the ability of one thing to enhance the effects of something else. For example, a purinosome dynamics modulator can enhance the effects of a therapeutic agent on a disease. The effects of the purinosome dynamics modulator and therapeutic agent together can be greater than the effect of each individually or the sum of the individual effects.

61. Test Molecule

A test molecule or like terms is a molecule which is used in a method to gain some information about the test molecule. A test molecule can be an unknown or a known molecule.

62. Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

63. Trigger

A trigger or like terms refers to the act of setting off or initiating an event, such as a response.

64. TBB

TBB is 4,5,6,7-tetrabromobenzotriazole.

65. TBBz

TBBz is 4,5,6,7-tetrabromobenzimidazole.

66. Therapeutic Efficacy

Therapeutic efficacy refers to the degree or extent of results from a treatment of a subject.

67. Unknown Molecule

An unknown molecule or like terms is a molecule with unknown biological/pharmacological/physiological/pathophysiological activity.

### EXAMPLES

### F. Material and Method

1. Materials

Epinephrine, clonidine, dopamine, acetylcholine, TBB (4,5,6,7-tetrabromobenzotriazole), DMAT (2-Dimethylamino-4,5,6,7-tetrabromo-1H-benzimidazole), TBBz (4,5,6,7-tetrabromobenzimidazole), DRB (5,6-Dichlorobenzimidazole 1-β-D-ribofuranoside), TBCA (Tetrabromocinnamic Acid), azaserine or hypoxanthine were obtained from Sigma Chemical Co. (St. Louis, Mo.). (Trp63, Trp64)-C3a(63-77), (Tyr65,Phe67)-C5a (65-74), agiotensin, bradykinin, cholecystokinin, corticotrophin-releasing factor, endothelin-1, galanin, ghrelin, glucagons, glucagon-like peptide, secretin, melanin-concentrating hormone, neuropeptide Y, neuropeptide B, neurotensin, dynorphin A, SFLLR-amide, SLIGKV-amide, substance P, urotensin II, (Arg8)-Vasopressin, vasoactive intestinal peptide, epidermal growth factor (EGF), fibroblast growth factor 1 (FGF1), hepatocyte growth factor (HGF), insulin-like growth factor 1(IGF1), nerve growth factor (NGF), platelet-derived growth factor (PDGFA), transforming growth factor beta (TGF-β), vascular endothelial growth factor (VEGF), and stem cell factor were obtained from Bachem (King of Prussia, Pa.). Epic® 384 biosensor microplates were obtained from Corning Inc. (Corning, N.Y.). Serotonin, Ro600175, adenosine, IB-MECA, A 61603, (R)-(−)-phenylephrine, (R)-(+)-m-nitrobiphenyline, ACEA, GABA (g-Aminobutyric acid), L-glutamate, L-serine-O-phosphate, L-aspartate acid, histamine, nicotinic acid, NPPB, 17-beta-estradiol, melatonin, ADP, ATP, UTP, UDP, epoprostenol, prostaglandin D2, peostaglandin E2, tyramine, 5-oxo-ETE, elaidic acid, yohimbine, LPA, sphingosine-1-phosphate, leukotriene B4, platelet activating factor PAF, Sphingosine-1-phosphate (S1P), lysophosphatidic Acid (LPA), ACEA, terbutaline, and oxymetazoline were obtained from Tocris (St. Louis, Mo.). Biomol adrenergic receptor ligand library (10 mM stock solutions in DMSO) was obtained from Enzo Life Science (Plymouth Meeting, Pa.), and used after dilution to desired concentrations.

2. Cell Culture

HeLa cells were obtained from American Type Cell Culture (Manassas, Va.). This cervical cancer cell line was maintained in regular serum medium (i.e., minimum essential medium (MEM) (Invitrogen) supplemented with 10% fetal bovine serum (FBS) and 1% Penicillin/streptomycin). To investigate the effects of purine depletion on the purinosome formation and dissociation, HeLa cells were maintained for at least two passages in “purine-depleted medium” prior cell assays. The purine depleted medium is a medium consisting of Roswell Park Memorial Institute 1640 (RPMI 1640) supplemented with dialyzed 5% FBS and. FBS was dialyzed against 0.9% NaCl at 4° C. for ˜2 days using a 25 kDa MWCO dialysis membrane to remove purines. This process completely removes the purines normally present in FBS.

All other cell lines were obtained from American Type Cell Culture (Manassas, Va.), were cultured under corresponding standard culture conditions, according to the protocol recommended by the supplier.

Cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. The confluency for all cells at the time of assays was ˜100%.

3. Optical Biosensor System and Cell Assays

Epic® wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. The compound solutions were introduced by using the on-board liquid handling unit (i.e., pippetting).

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, compound solutions were made by diluting the stored concentrated solutions with the HBSS (1× Hanks balanced salt solution, plus 20 mM Hepes, pH 7.1), and transferred into a 384 well polypropylene compound storage plate to prepare a compound source plate. Two compound source plates were made separately when a two-step assay was performed. In parallel, the cells were washed twice with the HBSS and maintained in 30 μl of the HBSS to prepare a cell assay plate. Both the cell assay plate and the compound source plate(s) were then incubated in the hotel of the reader system. After incubation the baseline wavelengths of all biosensors in the cell assay microplate were recorded and normalized to zero. Afterwards, a 2 to 10 min continuous recording was carried out to establish a baseline, and to ensure that the cells reached a steady state. Cellular responses were then triggered by transferring 10 μl of the compound solutions into the cell assay plate using the on-board liquid handler.

All studies were carried out at a controlled temperature (28° C.). At least two independent sets of experiments, each with at least three replicates, were performed. The assay coefficient of variation was found to be <10%.

4. Cell Transfection

The hFGAMS-GFP and hFGAMS-OFP constructs were prepared and used to transfect the before human cervical cancer cell line HeLa (An, S. et al. “Reversible compartmentalization of de novo purine biosynthetic complexes in living cells”. Science 2008, 320: 103-106). Briefly, HeLa cells were subjected to “purine-depleted medium”; Roswell Park Memorial Institute 1640 (RPMI 1640; Mediatech) supplemented with dialyzed 5% fetal bovine serum (FBS; Atlanta Biological) and 50 μg/mL gentamicin sulfate (Sigma), and “purine-rich medium”; Minimal Essential Medium (MEM; Mediatech) with 10% FBS and 50 μg/mL gentamicin sulfate. FBS was dialyzed against 0.9% NaCl at 4° C. for ˜2 days. Lipofectamine 2000 (Invitrogen) as a transfection reagent was used by following the manufacturer's protocol. Of note, an alternative purine-depleted medium (i.e. MEM, dialyzed 10% FBS and 50 μg/mL gentamicin sulfate) was evaluated with respect to purinosome formation in HeLa cells by transiently expressing hFGAMS-GFP.

5. Fluorescence Microscopy of Live and Fixed Cells

All samples were imaged at ambient temperature (˜25° C.) with a 60× objective (1.49 numerical aperture; Nikon Apo TIRF) using a Photometrics CoolSnap ES2 CCD detector mounted onto a Nikon TE-2000E inverted microscop. Oregon Green 488 and GFP detection was accomplished using a S484/15x excitation filter (Chroma Technology), S517/30m emission filter (Chroma Technology) and Q505LP/HQ510LP dichroic (Chroma Technology); rhodamine and OFP detection was carried out using a S555/25x excitation filter (Chroma Technology), S605/40m emission filter (Chroma Technology) and Q575LP/HQ585LP dichroic (Chroma Technology). Compounds were generally added to cells after three washes with buffered saline solution (20 mM HEPES (pH 7.4), 135 mM NaCl, 5 mM KCl, 1 mM MgCl₂, 1.8 mM CaCl₂ and 5.6 mM glucose). Cells transiently expressing hFGAMS-GFP were imaged with and without the compounds. Control experiments were also performed by addition of 4 μL DMSO.

### G. Results

1. CK2 Inhibitors as a Means to Regulate Purinosome Dynamics

The de novo purine biosynthetic pathway produces purines which represent the building blocks for DNA and RNA synthesis, provide energy in chemical and redox reactions, and act as signaling molecules in regulatory pathways. The de novo purine pathway consists of ten stepwise reactions that server to convert phosphoribosyl pyrophosphate to inosine monophosphate. In general, prokaryotes tend to use freestanding single-functional enzymes for the chemical transformation, while the higher eukaryotes rely on multifunctional enzymes in this pathway. Using confocal fluorescence imaging and transfection techniques, Benkovic and his colleagues (An, S., et al. Science 2008, 320, 103-106) found that all of these enzymes act within a multi-enzyme complex framework, and form a purinosome complex. Such purinosome complexes are dynamic and reversible, dependent on cellular conditions. Since label-free optical biosensor is sensitive to mass redistribution, the dynamic process of purinosome formation and dissembly could be directly monitored using the biosensor such as Epic® system. Also, since CK2 may play an important role in regulating purine synthesis pathway, CK2 inhibitors can be useful for detecting the dynamics of purinosome formation and dissembly.

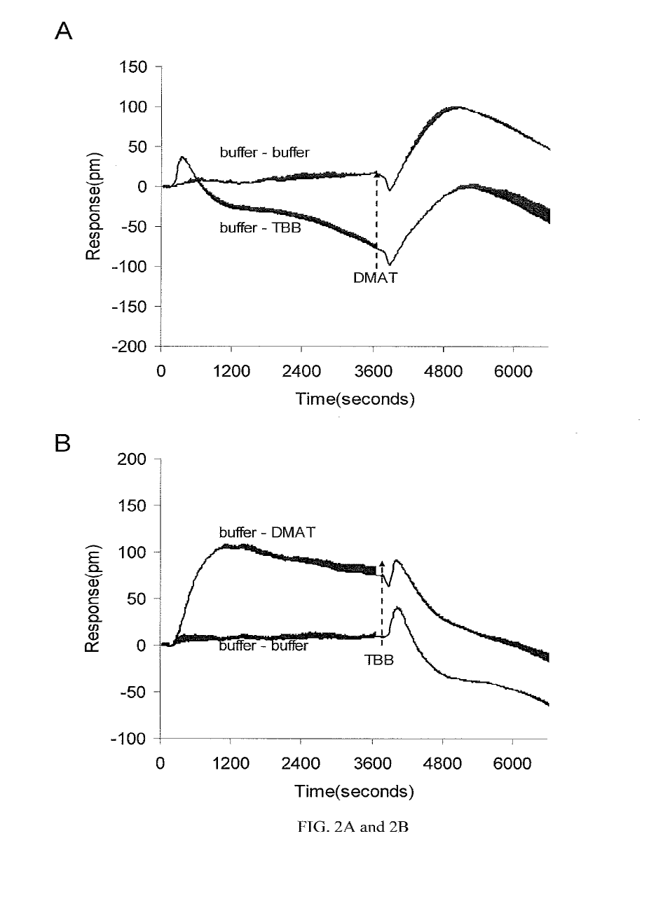

Since HeLa cells obtained using high initial seeding numbers of cells under regular serum culture medium responded to the two CK2 inhibitors differently, and both inhibitors led to robust DMR signals in the synchronized HeLa cells, the dynamics of DMR signals was examined. Results were summarized in FIG. 2. Here the cells were pre-treated with the assay vehicle only (i.e., buffer), followed by TBB and DMAT in a sequential order. Each step lasts about 1 hr. Results show that HeLa cells responded to TBB first with an N-DMR signal, and the pretreatment of cells with TBB did not alter the kinetics of the DMAT response, but slightly potentiated the DMAT response (FIG. 2A). However, when HeLa cells were simulated with DMAT first, the cells responded with a P-DMR signal, and the DMAT-treated cells further responded to TBB with N-DMR similar to the buffer treated cells (FIG. 2B). These results indicate that both CK2 inhibitors trigger a dynamic and reversible DMR signals in HeLa cells, and the two inhibitors display different modes of action.

Detailed pharmacology studies indicated that both inhibitors triggered DMR signals are saturable (data not shown). DMAT led to a dose-dependent and saturable P-DMR in the synchronized cells, leading to an EC50 of ˜5 to 22 μM, dependent on the time points after the DMAT stimulation. Similarly three other CK2 inhibitors, apigenin, DRB, and TBCA, led to DMR signals similar to the DMAT response. On the other hand, TBB also led to a dose-dependent and saturable DMR signal, but in the opposite direction (i.e., N-DMR), with an EC50 of 25 micromolar. Furthermore, the cells pretreated with TBCA or DRB became desensitized to the sequential DMAT stimulation, but only slightly potentiated the TBB response (data not shown). Taken together, these results indicate that label-free biosensor cellular assays are feasible to detect the CK2 activity in live cells, and can classify CK2 inhibitors into two types: purinosome promoting agent (e.g., DMAT, TBCA), and purinosome disruption agent (e.g., TBB, TBBz).

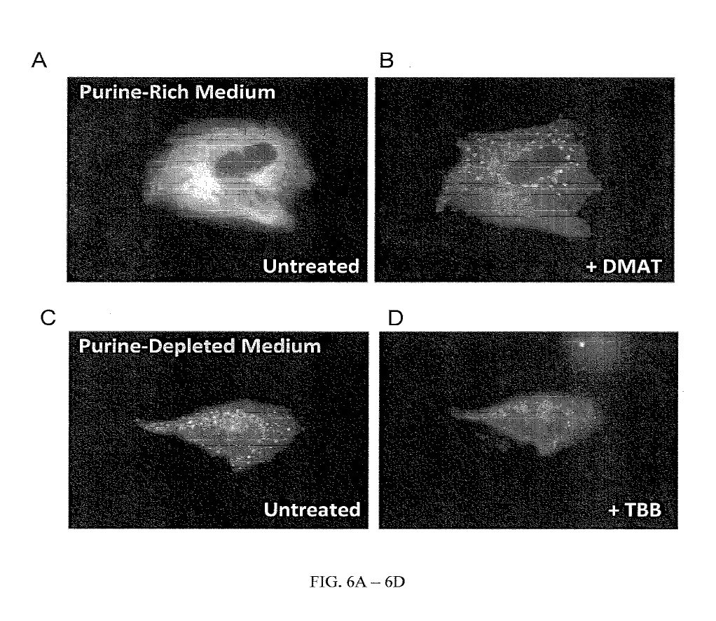

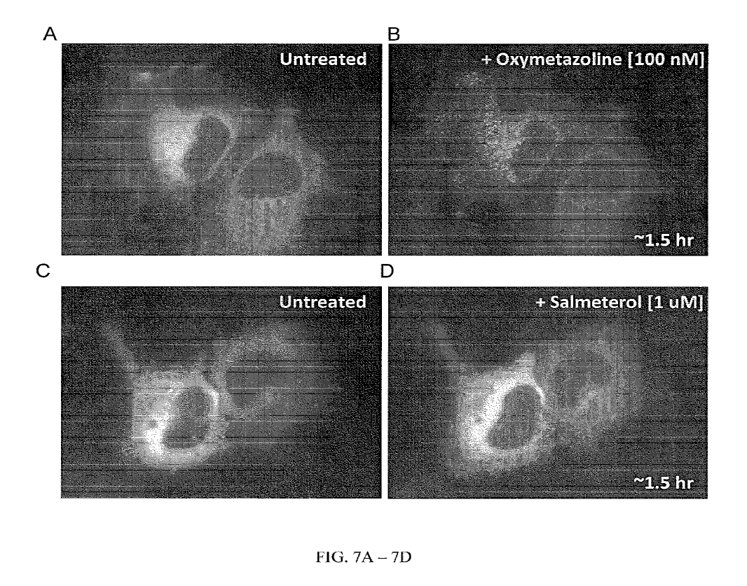

Confirmation using fluorescent imaging was made by stimulating cells, cultured under different conditions, with TBB or DMAT. An example was shown in FIG. 6. Here A549 cells were transfected with GFP-tagged hFGAMS using conventional transfection protocol, and cultured under two different conditions: purine rich medium and purine depleted medium. Under culture condition using the purine-rich medium, the GFP-tagged hFGAMS showed a diffusive pattern, indicating no or little purinosome complexes formed (FIG. 6A). Stimulation of the A549 cells with DMAT resulted in the appearance of fluorescent clusters, indicating the formation of purinosome complexes (FIG. 6B). Conversely under purine depleted culture condition, A549 cells transiently transfected with the GFP-tagged hFGAMS gave rise to many fluorescence clusters, indicating the formation of many purinosome complexes (FIG. 6C). Stimulation of this A549 cell with TBB resulted in the disappearance of fluorescent clusters, indicating the dissociation of purinosome complexes (FIG. 6D). Similar results were observed in Hela cells and A431 cells (data not shown), indicating that the CK2 inhibitor regulated purinosome dynamics is general to different types of cells. These results are consistent with, thus confirmative to, the results obtained using label-free cellular assays.

To further confirm the specificity of the CK2 inhibitor induced purinosome dynamics, RNAi knockdown of CK2 enzyme in Hela was carried out using conventional RNAi knockdown protocols. Results showed that compared to mock or scrumble RNAi transfection controls, CK2 knockdown suppressed both TBB and DMAT induced DMR signals in Hela cells (˜25% suppression, a level was comparable to the total knockdown level of CK2 enzymes, as examined using western blotting) (data not shown). These results indicate that both TBB and DMAT induced DMR signals are largely due to their modulation of endogenous CK2 enzyme activity, and DMAT promotes purinosome formation while TBB suppresses the purinosome formation.

2. Screening Cellular Targets that are Involved in Regulating Purinosome Dynamics

The upstream signaling pathways linking to the purinosome is unknown at the present time. To identify cellular targets and pathways that are involved in regulating purinosome dynamics, a library of agonists for a great number of G protein-coupled receptors and membrane bound receptor tyrosine kinases was made in house, and each agonist was used to stimulate a confluent layer of Hela cells, cultured using high initial seeding number (25k per well for 384 well Epic® cell culture compatible biosensor microplates) in purine rich medium (i.e., regular 10% FCS or FBS). The concentrations for all small organic molecules were 10 micromolar, whereas 1 micromolar for all small peptides, and 100 nanomolar for all protein factors (molecular weight >15k Da). The cellular responses to each agonist were recorded for about 1 hour, and used to determine the functional expression of their corresponding cognate receptor(s). The receptor specificity was further confirmed by real time PCR. RT-PCR showed that Hela cells endogenously express 5-hydroxytryptamine receptor 1D (5HT1D), adenosine A2b receptor, alpha2A-adrenergic receptor (alpha2A AR), beta2-adrenergic receptor (beta2AR), bradykinin receptor B1 (BRAD1), coagulation factor II (thrombin) receptor PAR1 (low), glucagon-like peptide 2 receptor, histamine receptor 1 (H1R), lysophosphatidic acid receptor 1, 2, and 5 (all at low level), prostaglandin E receptor 4 (EP4), purinergic receptor P2Y2, somatostatin receptor 1, sphingosine-1-phosphate receptor 2, 3 and 5 (S1P2, S1P3, and S1P5, respectively; S1P3 being dominate isoform). This expression pattern at mRNA level is largely consistent with the functional readout, as measured as DMR signals by Epic® cellular assays (data not shown).

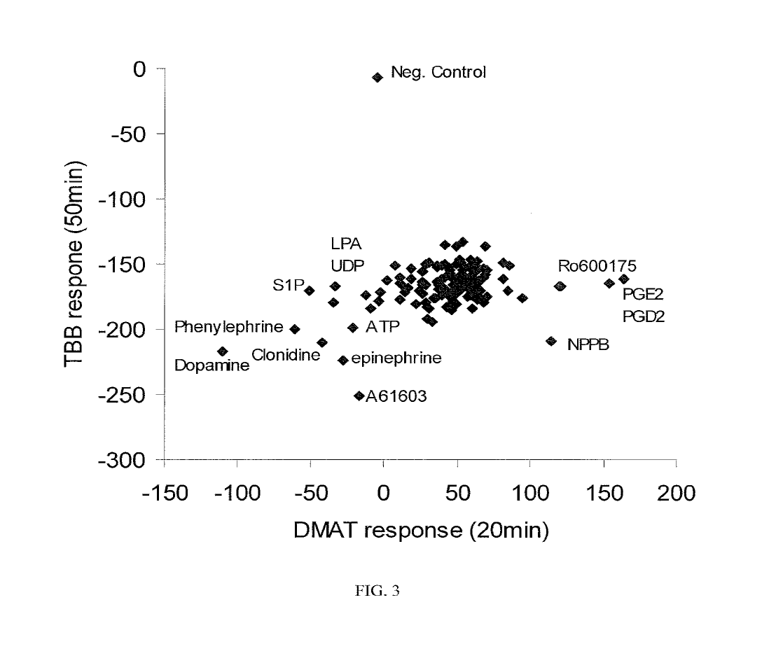

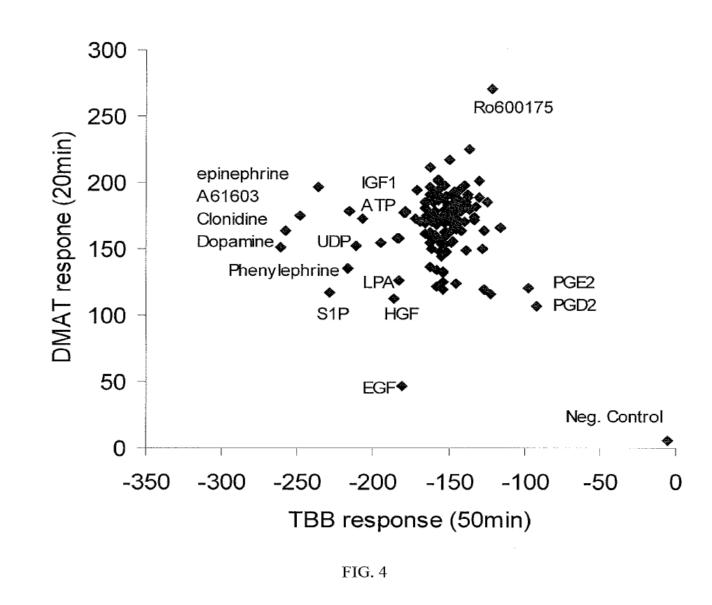

To examine the receptor whose signaling is involved in regulating the purinosome dynamics, Hela cells were pretreated individually with each agonist for about one hour, followed by sequential stimulation with DMAT (20 micromolar) and TBB (25 micromolar), each step about 1 hour. The DMR signals of each step were monitored separately. The amplitude of each DMR signal was calculated, as indicated in FIG. 3 and FIG. 4. In the first experiment wherein the sequential stimulation order is agonist, DMAT and TBB, the correlation analysis showed that these agonists differently affected both DMAT and TBB DMR signals (FIG. 3). Four types of agonists can be identified from the correlation plot—(1) the first set of agonists that have no or little impact on both DMR signals (non-effectors); (2) the second set of agonists that have no or little impact on the TBB DMR signal, but potentiate the DMAT DMR signals (examples are the EP4 agonist PGE2 and PGD2, the 5HT1D agonist Ro600175, and an ion channel modulator NPPB); (3) the third set of agonists that selectively suppress the DMAT DMR signal (examples are the LPA1, 2, and 5 agonist LPA, the P2Y2 agonist UDP and ATP, and the S1P2, 3, and 5 agonist S1P); and (4) the fourth set of agonist that not only suppress the DMAT signal but also potentiate the TBB signal (examples are adrenergic receptor agonists phenylephrine, dopamine, clonidine, epinephrine and A61603).

In the second experiment wherein the sequential stimulation order is agonist, TBB and DMAT, the correlation analysis showed that these agonists also differently affected both DMAT and TBB DMR signals (FIG. 4). Five types of agonists can be identified from the correlation plot—(1) the first set of agonists that have no or little impact on both DMR signals (non-effectors); (2) the second set of agonists that selectively suppress the TBB DMR signal (examples are the EP4 agonist PGE2 and PGD2); (3) the third set of agonists that selectively potentiate the DMAT signal (the 5HT1D agonist Ro600175); (4) the fourth set of agonists that selectively potentiate the TBB signal (examples are the LPA1, 2, and 5 agonist LPA, the P2Y2 agonist UDP and ATP, the S1P2, 3, and 5 agonist S1P, the IGF1R agonist IGF1, the HGFR agonist HGF, and the adrenergic receptor agonists phenylephrine, dopamine, clonidine, epinephrine and A61603); and (5) the fifth set of agonists that selectively suppress the DMAT signal (an example is the EGFR agonist EGF). It is worth noting that for these non-effector agonists, their respective receptors were found to be not expressed in Hela cells, using RT-PCR.

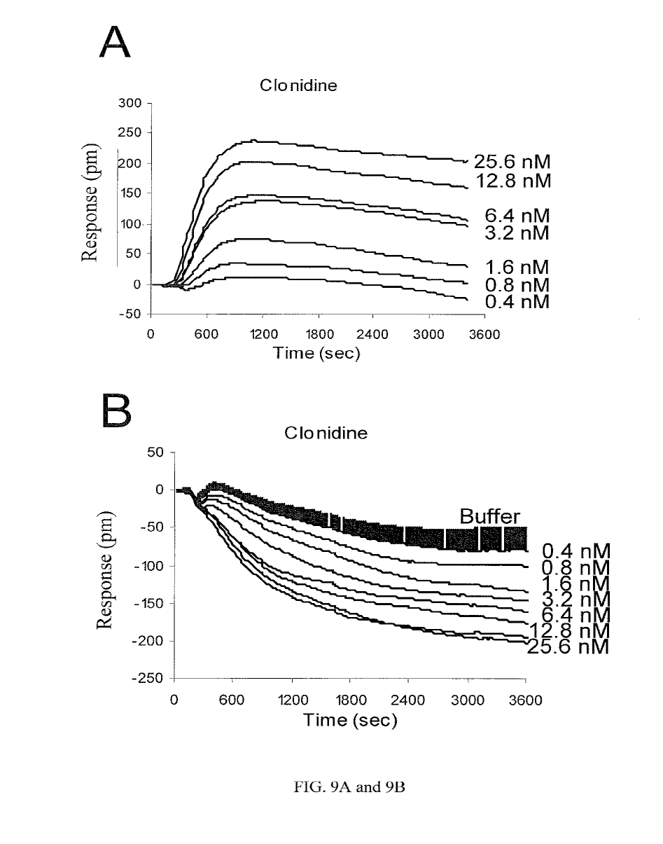

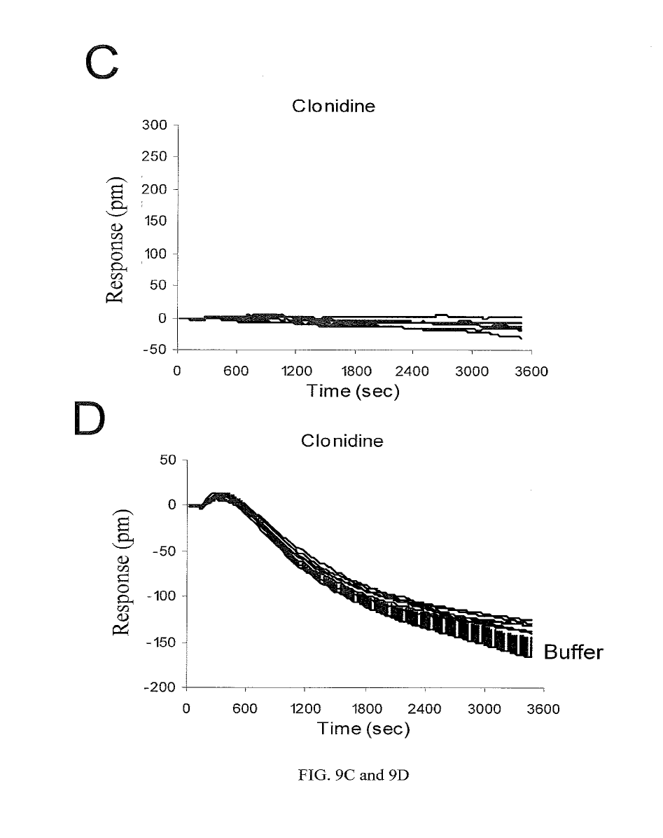

These results indicate that many different types of receptors act as an upstream signaling cascade of the purinosome dynamics, and different receptor signaling regulates differently the purinosome dynamics. Interestingly, the four adrenergic receptor agonists all behaved similarly—they can trigger a positive DMR signal in Hela cells (exampled in FIG. 9); they can suppress the DMAT signal but potentiate the TBB signal in the purinosome formation and dissociation dynamics (FIG. 3); and they can selectively potentiate the TBB signal in the purinosome dissociation and formation dynamics (FIG. 4 and FIG. 9). These results indicate that these 4 agonists activate an endogenous receptor, whose signaling promotes the formation of purinosome complexes, thus desensitizing the DMAT signal, but potentiating the TBB signal.

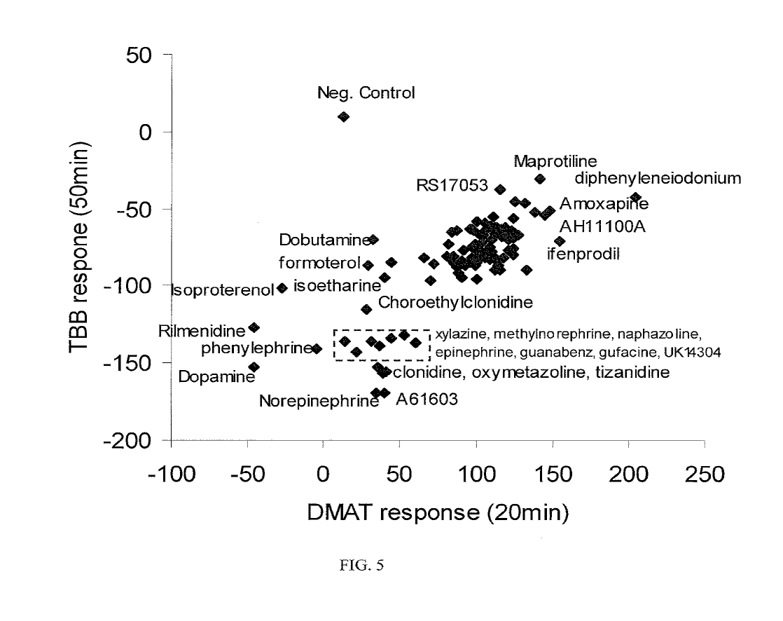

Since Hela cell endogenously expresses both alpha2A-AR and beta2-AR, these adrenergic receptors may activate both receptors. A library of adrenergic receptor ligands was further examined for their ability to modulate the purinosome dynamics Results were summarized in FIG. 5. These AR ligands can be classified into four types: (1) non-effectors; (2) ligands that potentiate the DMAT signal but suppress the TBB signal (ifenprodil, AH11110A, RS17053, amoxapine, diphylpheneiodonium, and maprotiline; all of which are alpha2-selectively antagonists); (3) ligands that specifically suppress the DMAT signal (dobutamine, formoterol, isoetharine, and isoproterenol; all of which are beta2 agonists); (4) ligands that suppress the DMAT signal and potentiate the TBB signal (rilmenidine, phenylephrine, dopamine, norepinephrine, A61603, clonidine, oxymetazoline, tizanidine, xylazine, methylnorephrine, naphazoline, epinephrine, guanabenz, gufacine, and UK14304; most of which are alpha2 selectively agonists, except for dopamine, norepinephrine, epinephrine and methylnorephrine which are non-selective to adrenergic receptors). Taken together, these results indicate that it is alpha2A receptor whose signaling leads to the formation of purinosome complexes.

To confirm the linkage of alpha2A, but not beta2AR, to purinosome formation, fluorescence imaging was used. Here Hela cells were transfected with GFP-tagged hFGAMS, an enzyme involved in purine synthesis pathway and purinosome complex. Under purine rich culture conditions, the fluorescence pattern of GFP-hFGAMS is diffusive, indicating that there is no or little purinosome complexes formed. However, the treatment of the cells with the alpha2AR specific agonist oxymetazoline resulted in the formation of many fluorescence clusters. Conversely, the stimulation of Hela cells with the beta2-AR specific agonist salmeterol did not result in the formation of fluorescent clusters. These results confirm that the alpha2A-AR signaling, but not the beta2AR signaling, is a promoter of purinosome complexes.

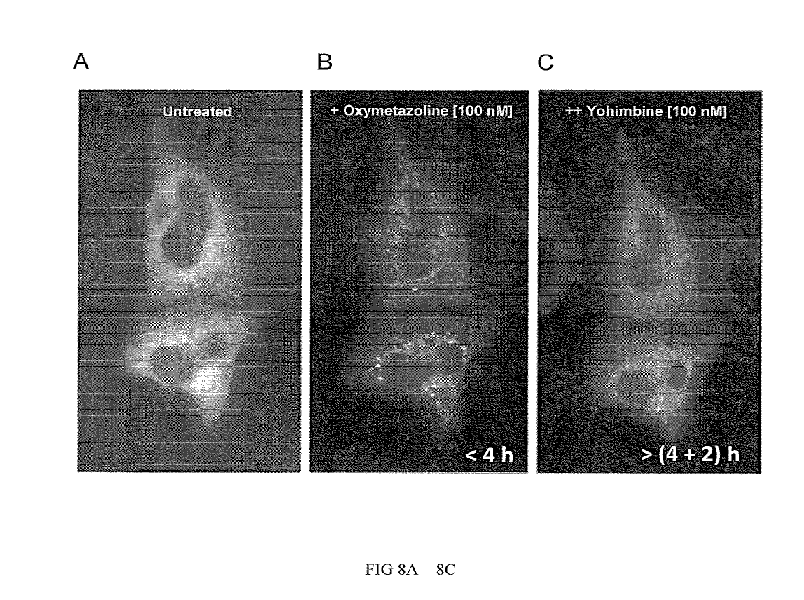

The biosensor screen data shown in FIG. 5 also indicate that certain alpha2A-AR antagonist can suppress the purinosome formation or even cause the dissociation of purinosome complexes. To test this, fluorescence imaging of GFP-hFGAMS in Hela cells were used. The main results were summarized in FIG. 8. Again, the stimulation of Hela cells cultured under purine rich medium with oxymetazoline caused the appearance of fluorescent clusters (i.e., purinosome formation). Interestingly, the subsequent stimulation of the oxymetazoline-incubated cells with the alpha2 selective antagonist yohimbine caused the dissociation of the purinosome complexes. This is significant in two different aspects: (1) the receptor antagonist can reverse the signal event downstream the receptor after the receptor becomes fully activated; and (2) the purinosome formation driven by the activation of alpha2A-AR is a downstream cellular event, possibly after receptor internalization, but before de novo synthesis and gene expression—indicating that the purinosome formation may be an immediate and early response in GPCR mitogenic signaling.

3. Gi Mediated Signaling is Linked to the Alpha2A-AR with the Purinosome Formation

To further elucidate the signaling pathways linking the alpha2A AR signaling to the purinosome dynamics, pertussis toxin (PTX) was used to kill the Gi protein in Hela cells. Results showed that the alpha2 agonist clonidine triggered a dose-dependent and saturable DMR signal in Hela (FIG. 9A), and resulted in a dose-dependent potentiation of the TBB response (FIG. 9B). However, the pretreatment of Hela cells with PTX of 100 ng/ml completely suppressed the clonidine signal within the concentration range of clonidine examined (FIG. 9C), and clonidine did not result in any significant change in the TBB signal of the PTX-treated Hela cells (FIG. 9D). Similarly, the PTX treatment only partially suppressed the epinephrine DMR signal (data not shown), suggesting that epinephrine acts as a non-selective agonist and activates both alpha2A and beta2-AR in Hela cells. Alpha2A is a Gi-coupled receptor, whereas beta2-AR is a Gs-coupled receptor. These results further confirm that it is alpha2A, but not beta2AR, that is involved in regulation of purinosome dynamics via Gi-mediated signaling in Hela cells.

### REFERENCES

U.S. Application US2006/013539. Fang, Y., Ferrie, A. M., Fontaine, N. M., Yuen, P. K. and Lahiri, J. “Optical biosensors and cells”
U.S. application Ser. No. 12/708,840. Fang, Y., and Verrier, F. Methods related to casein kinase II (CK2) inhibitors and the use of purinosome disrupting CK2 inhibitors for anti-cancer therapy agents.
An, S. et al. “Reversible compartmentalization of de novo purine biosynthetic complexes in living cells”. Science 2008, 320: 103-106

We claim:

1. A method of identifying cellular targets involved in regulating purinosome dynamics comprising
a) providing a cell;
b) contacting the cell having a known cellular target with a molecule having a known cellular target forming a molecule incubated cell;
c) contacting the molecule incubated cell sequentially with a purinosome promoting agent and a purinosome disrupting agent;
d) monitoring the response of the molecule incubated cell after contact with the purinosome promoting agent and after contact with the purinosome disrupting agent;
e) determining the ability of the molecule to modulate the dynamics of the purinosome formation and dissociation.

2. The method of claim 1, wherein the purinosome promoting agent contacts the cell before the purinosome disrupting agent.

3. The method of claim 1, wherein the purinosome disrupting agent contacts the cell before the purinosome promoting agent.

4. The method of any one of claim 1, further comprising classifying the cellular targets based on their regulation of purinosome dynamics.

5. The method of claim 4, wherein the classifying of cellular targets is based on correlation analysis.

6. The method of any one of claim 1, wherein the cellular target is a G protein-coupled receptor (GPCR), a receptor tyrosine kinase, a Toll-like receptor, a cytokine receptor, or ion channel.

7. The method of claim 6, wherein the GPCR is a prostaglandin receptor, serotonin receptor, adrenergic receptor (AR), lysophosphatidic acid (LPA) receptor, P2Y2 receptor, or a sphingosine 1-phosphate (S1P) receptor.

8. A method of identifying a purinosome dynamics modulating pathway comprising
a) providing a cell;
b) contacting the cell having a known cellular target with a cellular pathway modulator specific to the cellular pathway of said cellular target to obtain a cellular pathway modulator incubated cell.
c) contacting the cellular pathway modulator incubated cell with a ligand specific to said cellular target to obtain the cellular pathway modulator and ligand incubated cell;
d) contacting said cellular pathway modulator and ligand incubated cell with a purinosome modulating agent;
e) assaying the response of the cell; and
f) determining the ability of the cellular pathway modulator to regulate purinosome dynamics, wherein the ability of the cellular pathway modulator to regulate purinosome dynamics indicates the cellular pathway is a purinosome dynamics modulating pathway.

9. The method of claim 8, wherein the ligand is an agonist.

10. The method of claim 8, wherein the purinosome modulating agent is a purinosome disrupting agent.

11. A method of identifying purinosome dynamics modulators comprising
a) providing a cell;
b) contacting the cell with a molecule forming a molecule incubated cell;
c) contacting the molecule incubated cell sequentially with a purinosome promoting agent and a purinosome disrupting agent;
d) monitoring the response of the molecule incubated cell after contact with the purinosome promoting agent and after contact with the purinosome disrupting agent;
e) determining the ability of the molecule to modulate the dynamics of the purinosome formation and dissociation.

12. The method of claim 11, wherein the purinosome promoting agent contacts the cell before the purinosome disrupting agent.

13. The method of claim 11, wherein the purinosome disrupting agent contacts the cell before the purinosome promoting agent.

14. The method of any one of claim 11, further comprising classifying the purinosome dynamics modulator based on its regulation of purinosome dynamics.

15. The method of claim 14, wherein the classifying of the purinosome dynamics modulator is based on similarity analysis.

16. A method of treating a subject comprising administering to the subject a therapeutically effective amount of a purinosome dynamics modulator, wherein the subject has a disease which is pathophysiologically related to purinosomes.

17. The method of claim 16, further comprising one or more therapeutic agents.

18. The method of claims 17, wherein the therapeutic agent is a protein kinase inhibitor.

19. The method of claim 18, wherein the protein kinase inhibitor is selected from erlotinib, lapatinib, dasatinib, temsirolimus, rapamycin, sorafenib, or sunitinib

20. A pharmaceutical composition for treating a subject comprising a therapeutically effective amount of a purinosome dynamics modulating molecule.
